(19) United States
(12) Patent Application Publication
(10) Pub. No.: US 2026/0033968 A1
(43) Pub. Date: Feb. 5, 2026

(54) FOLDING MECHANISM FOR AN INTRODUCER SHEATH

(71) Applicant: EDWARDS LIFESCIENCES CORPORATION, Irvine, CA (US)

(72) Inventors: Haim Yushtein, Netanya (IL); David Maimon, Atlit (IL); Yair A. Neumann, Moshav Sede Varburg (IL)

(73) Assignee: EDWARDS LIFESCIENCES CORPORATION, Irvine, CA (US)

(21) Appl. No.: 19/356,540

(22) Filed: Oct. 13, 2025

Related U.S. Application Data
(63) Continuation of application No. PCT/US2024/024478, filed on Apr. 12, 2024.
(60) Provisional application No. 63/459,522, filed on Apr. 14, 2023.

(51) Int. Cl. A61F 2/95; A61F 2/24

(52) U.S. Cl. CPC A61F 2/9526; A61F 2/2427; A61F 2/9524

(57) ABSTRACT

A folding device for an introducer sheath is disclosed herein. The folding device includes a housing including a plurality of jaws rotatably coupled to the housing, each of the plurality of jaws is rotatable within a corresponding channel extending through a side wall of the housing. Each of the plurality of jaws are rotatable between a first (retracted) position, where the plurality of jaws do not extend into a central opening of the housing, and a second (contracted) position where at least a portion of each of the plurality of jaws extends into the central opening of the housing in a configuration to fold and/or compress a portion of the sheath received within the central opening of the housing.

### CROSS-REFERENCE TO RELATED APPLICATIONS

This application is a continuation of International Application No. PCT/US2024/024478, filed Apr. 12, 2024, which claims the benefit of U.S. Provisional Application No. 63/459,522, filed Apr. 14, 2023, the contents of which are incorporated herein by reference in its entirety.

### FIELD

The present application relates to expandable introducer sheaths for prosthetic devices such as transcatheter heart valves, and methods of making the same.

### BACKGROUND

Endovascular delivery catheter assemblies are used to implant prosthetic devices, such as a prosthetic valve, at locations inside the body that are not readily accessible by surgery or where access without invasive surgery is desirable. For example, aortic, mitral, tricuspid, and/or pulmonary prosthetic valves can be delivered to a treatment site using minimally invasive surgical techniques.

An introducer sheath can be used to safely introduce a delivery apparatus into a patient's vasculature (for example, the femoral artery). An introducer sheath generally has an elongated sleeve that is inserted into the vasculature and a housing that contains one or more sealing valves that allow a delivery apparatus to be placed in fluid communication with the vasculature with minimal blood loss. Once the introducer sheath is positioned within the vasculature, the shaft of the delivery apparatus is advanced through the sheath and into the vasculature, carrying the prosthetic device.

Such introducer sheaths may be radially expandable. The sheath, formed of highly elastomeric materials and some including one or more folds to aid in expansion, expands as the delivery apparatus/implantable device is inserted through the sheath. Example sheaths include complex mechanisms, such as ratcheting mechanisms that maintain the sheath in an expanded configuration once a device with a larger diameter than the sheath's original diameter is introduced. Existing expandable sheaths can also be prone to axial elongation as a consequence of the application of longitudinal force attendant to passing a prosthetic device through the sheath. Such elongation can cause a corresponding reduction in the diameter of the sheath, increasing the force required to insert the prosthetic device through the narrowed sheath.

Accordingly, there remains a need in the art for an improved introducer sheath for endovascular systems used for implanting valves and other prosthetic devices.

### SUMMARY

The expandable sheaths disclosed herein include a first polymeric layer and a braided layer positioned radially outward of the first polymeric layer. The braided layer includes a plurality of filaments braided together. The expandable sheaths further include a resilient elastic layer positioned radially outward of the braided layer. The elastic layer is configured to apply radial force to the braided layer and the first polymeric layer. The expandable sheaths disclosed herein further include a second polymeric layer positioned radially outward of the elastic layer and bonded to the first polymeric layer such that the braided layer and the elastic layer are encapsulated between the first and second polymeric layers. When a medical device is passed through the sheath, the diameter of the sheath expands from a first diameter to a second diameter around the medical device while the first and second polymeric layers resist axial elongation of the sheath such that a length of the sheath remains substantially constant. The sheath resiliently returns to the first diameter by radial force applied by the elastic layer upon passage of the medical device. In some examples, the first and second polymeric layers include a plurality of longitudinally-extending folds when the sheath is at the first diameter. The longitudinally extending folds create a plurality of circumferentially spaced ridges and a plurality of circumferentially spaced valleys. As a medical device is passed through the sheath, the ridges and valleys level out to allow the sheath to radially expand. The elastic layer can include one or more elastic bands helically wound over the braided layer. In some examples, two elastic bands are wound with opposite helicity. As noted herein, the braided layer is positioned radially outward of the first polymeric layer, but radially inward of the second polymeric layer. The filaments of the braided layer can be movable between the first and second polymeric layers such that the braided layer can radially expand as a medical device is passed through the sheath while the length of the sheath remains substantially constant, and, in some examples, the filaments of the braided layer are not engaged or adhered to the first or second polymeric layers at all. The filaments of the braided layer can also be resiliently buckled when the sheath is at the first diameter. In this example, the first and second polymeric layers can be attached to each other at a plurality of open spaces between the filaments of the braided layer. Some examples can include one or more longitudinally extending cords attached to the braided layer. An outer cover can extend longitudinally beyond the distal ends of the first polymeric layer, the braided layer, the elastic layer, and the second polymeric layer to form an overhang. In some examples, the outer cover comprises one or more longitudinally extending slits, weakened portions, or scorelines. In some examples, the outer cover is formed of a heat-shrink material. In some examples, the outer cover is elastomeric.

A folding device for an introducer sheath is disclosed herein. In one of its basic configurations, the folding device includes housing and a plurality of jaws configured to fold and/or compress a portion of an expandable sheath. The basic configuration can preferably be provided with any one or more of the features described herein, in particular with those of the examples described hereafter. However, it should be understood that the basic configuration can preferably also be provided with any one or more of the features shown in the figures and/or described in conjunction with the figures, either in addition to or alternatively to the features of the examples described hereafter.

In some examples, the housing includes a central opening extending therethrough and a plurality of channels extending through a side wall of the housing, the plurality of channels positioned circumferentially around the housing.

In some examples, the plurality of jaws are rotatably coupled to the housing and rotatable within a corresponding one of the plurality of channels. Each of the plurality of jaws are rotatable between a first (retracted) position, where the plurality of jaws do not extend into the central opening of the housing, and a second (contracted) position where at least a portion of each of the plurality of jaws extends into the central opening of the housing in a configuration to fold and/or compress a portion of the sheath received within the central opening of the housing.

In some examples, the central opening is sized and configured to receive an unfolded and/or uncompressed portion of an introducer sheath (for example, an unfolded and/or uncompressed distal end portion of the sheath) when the plurality of jaws are in the first (retracted) position.

In some examples, the folding device is sized and configured to fold and/or collapse a circumference of a portion (for example, a distal end portion) of a sheath (for example, an introducer sheath) positioned within the central opening of the housing when the plurality of jaws are moved from the first position to the second position such that symmetrical folds are formed around a circumference of the sheath.

In some examples, each of the plurality of jaws is rotatably coupled to the housing at a corresponding axel extending between a proximal end and distal end of the housing, each of the axels extending through a jaw axel bore provided in each of the plurality of jaws.

In some examples, each of the axels extend through the housing at a corresponding housing axel bore.

In some examples, the housing includes: a proximal end portion extending between a proximal end surface of the housing and a proximal end surface of each of the plurality of channels; a distal end portion extending between a distal end surface of the housing and a distal end surface of each of the plurality of channels, where each of the housing axel bores extend through the proximal and distal end portions of the housing.

In some examples, each of the plurality of jaws includes a first (leading) inner surface, a second (trailing) inner surface, and an outer surface, where the second (trailing) inner surface is located between the first (leading) inner surface and the outer surface.

In some examples, the first (leading) inner surface defines a concave surface and the second (trailing) inner surface defines a convex surface.

In some examples, a curvature of the outer surface corresponds with an outer curvature of the housing.

In some examples, when the plurality of jaws are in the first position, the first (leading) inner surface is positioned proximate (for example, adjacent, aligned with) the central opening.

In some examples, as the plurality of jaws move from the first position to the second position, the first (leading) inner surface and the second (trailing) inner surface extend into the central opening of the housing.

In some examples, as the plurality of jaws move from the first position to the second position, each of the plurality of jaws are displaced inward forming an opening (for example, crescent-shaped opening) between adjacent jaws (for example, configured to capture portions of the inner and outer layer (for example, Dyneema® layers) there-between during the folding procedure and thereby creating the folded portions of the sheath disposed around its circumference in an orderly and symmetrical manner).

In some examples, movement of the plurality of jaws to the second position results from displacing the plurality of jaws radially inward.

In some examples, each of the plurality of jaws are movable between the first and second position in a coordinated manner.

In some examples, the folding device further includes a mandrel sized and configured to be received within the central opening when the plurality of jaws are in the second position.

In some examples, a portion of the mandrel has a star-shaped cross section.

In some examples, the portion of the mandrel including the star-shaped cross section includes crescent-shaped wings extending at an angle radially outward from a body portion of the mandrel (for example, where the crescent-shaped wings are complementary to the crescent/star-shaped opening defined by the inner surfaces of the jaws during folding/compression of the sheath).

In some examples, the folding device further includes a second mandrel sized and configured to be received with the central opening, the second mandrel having a diameter less than a diameter of the mandrel and having a circular-shaped cross section; and a third mandrel sized and configured to be received within the central opening, the third mandrel having a diameter less than a diameter of the second mandrel and having a circular-shaped cross section.

In some examples, the folding device further includes a crimping mechanism (for example, conventional crimping mechanism) for directing movement of the plurality of jaws between the first position and the section position, wherein the housing is received within the crimping mechanism.

In some examples, the crimping mechanism includes a plurality of jaws movable to contract and expand a diameter of a compression channel provided within the crimping mechanism, wherein the housing is received within the compression channel such that expansion and contraction of the diameter of the compression channel drives the corresponding movement of the plurality of jaws between the first and second position.

A method of folding a sheath is disclosed herein. In some examples, the method includes forming symmetrical folds around the distal end portion of the sheath. In some examples, the method includes inserting a portion of an uncompressed sheath (for example, the distal end portion of an uncompressed sheath) into a folding device where the folding device includes: a housing including a central opening extending therethrough and a plurality of channels extending through a side wall of the housing, the plurality of channels positioned circumferentially around the housing; and a plurality of jaws rotatably coupled to the housing, each of the plurality of jaws is rotatable within a corresponding one of the plurality of channels, where each of the plurality of jaws are rotatable between a first (retracted) position where the plurality of jaws do not extend into the central opening of the housing and a second (contracted) position where at least a portion of each of the plurality of jaws extends into the central opening of the housing.

In some examples, the method further includes moving the plurality of jaws from the first position toward the second position (for example, partially compressing the plurality of jaws) by rotating each of the plurality of jaws radially inward such that the portion of the uncompressed sheath is captured between adjacent jaws thereby creating the folded portions of the sheath and forming a compressed distal end portion of the sheath.

In some examples, the plurality of jaws are moved from the first position toward the second position in a coordinated manner.

In some examples, the plurality of jaws are moved from the first position toward the second position in response to a radially inward force provided against an outer surface of the jaws.

In some examples, the method further includes: inserting a mandrel within a central lumen of the uncompressed sheath (for example, after the distal end portion of the uncompressed sheath is inserted into a folding device), the mandrel having a star-shaped cross section including a plurality of wings (for example, crescent-shaped wings) extending radially outward from a body portion of the mandrel; and moving the plurality of jaws from the first (retracted) position toward the second (contracted) position by rotating each of the plurality of jaws radially inward such the portion of the uncompressed sheath is captured between adjacent jaws and compressed against the plurality of wings provided on the mandrel, thereby creating folded portions of the sheath disposed around its circumference and forming the compressed portion (for example, a folded and compressed portion) of the sheath (for example, of the distal end portion of the sheath).

In some examples, in the second position, the plurality of wings define a shape complementary to the star-shaped opening between the plurality of jaws.

In some examples, the method further includes: withdrawing the mandrel from the central lumen of the compressed portion of the sheath (for example, the compressed distal end portion); and inserting a second mandrel within the central lumen of the compressed portion of the sheath, the second mandrel having a diameter less than a diameter of the mandrel.

In some examples, the method further includes moving the plurality of jaws toward the second (contracted) position by rotating each of the plurality of jaws radially inward such that the folded portions of the sheath are further compressed against an outer surface of the second mandrel.

In some examples, the method further includes: removing the sheath from the central lumen of the folding device and withdrawing the second mandrel from the central lumen of the portion (for example, distal end portion) of the sheath; and further compressing the compressed portion (for example, compressed distal end portion) of the sheath.

In some examples, the method further includes: inserting the compressed portion of the sheath into the central lumen of the folding device; and further compressing the compressed portion of the sheath by moving the plurality of jaws toward the second position.

In some examples, the sheath is provided over a third mandrel (for example, the third mandrel is used throughout the process to internally support the layers of the sheath (for example, the inner and outer layers composed of Dyneema®) during compression/folding/pleating, utilization of the third mandrel at the final stages will support the internal circular shape of the compressed tip), where further compressing the compressed portion (for example, compressed distal end portion) of the sheath includes compressing the folded portions against an outer surface of the third mandrel.

In some examples, compression of the folded portions against the outer surface of the third mandrel results in plastic deformation of the folded portions of the sheath and provides a laid-over configuration of the folded layers of the folded portions.

In some examples, the compressed portion (for example, compressed distal end portion) of the sheath is further compressed using a crimping mechanism, where further compressing the compressed portion (for example, compressed distal end portion) of the sheath includes inserting the compressed portion of the sheath within a compression channel of the crimping mechanism such that expansion and contraction of a diameter of the compression channel drives the corresponding movement of the plurality of jaws between the first and second position.

In some examples, the housing is received within a compression channel of a crimping mechanism such that expansion and contraction of a diameter of the compression channel drives the corresponding movement of the plurality of jaws between the first and second position.

In some examples, the method further includes applying a heat treatment to the compressed portion (for example, compressed distal end portion) of the sheath to result in the final desired tip-shape.

In some examples, the compressed portion (for example, compressed distal end portion) of the sheath includes at least three folded portions (for example, where the number of folded portions included on the sheath is determined by the number of jaws and the corresponding number wings provided on the mandrel, in some implementations the sheath includes three, four or five folded portions).

In some examples, the method further includes: sealing a distal end of the uncompressed sheath, forming a sealed portion; coupling a compressed air and vacuum device to the proximal end of the uncompressed sheath; applying a positive pressure from the compressed air and vacuum device against an internal surface of the sheath; moving the plurality of jaws toward the second position by rotating each of the plurality of jaws radially inward thereby creating partially folded portions of the sheath (for example, first folding step, with star-shaped mandrel); and applying a negative pressure against the internal surface of the partially compressed sheath and further moving the plurality of jaws toward the second position by rotating each of the plurality of jaws radially inward thereby creating the folded portions of the sheath and forming the compressed portion of the sheath (for example, second folding step, with second mandrel).

In some examples, the positive pressure is applied against the internal surface of the sheath before the plurality of jaws are moved from the first position toward the second position.

In some examples, the positive pressure is applied against the internal surface of the sheath after the plurality of jaws have partially moved from the first position toward the second position and the folded portions of the sheath have been partially formed.

In some examples, the method further includes: further compressing the compressed portion of the sheath by moving the plurality of jaws toward the second position such that the folded portions plastically deform in a laid-over configuration; applying a heat treatment to the compressed portion of the sheath; and removing the sealed portion from the distal end of the sheath.

Various aspects of the examples described above can be combined based on desired sheath system characteristics.

### BRIEF DESCRIPTION OF THE DRAWINGS

FIG. 1 illustrates a delivery system for a cardiovascular prosthetic device, according to one implementation.

FIG. 2 illustrates an expandable sheath that can be used in combination with the delivery system of FIG. 1, according to one implementation.

FIG. 3 is a magnified view of a portion of the expandable sheath of FIG. 2.

FIG. 4 is a side elevation cross-sectional view of a portion of the expandable sheath of FIG. 2.

FIG. 5A is a magnified view of a portion of the expandable sheath of FIG. 2 with the outer layer removed for purposes of illustration.

FIG. 5B is a magnified view of a portion of the braided layer of the sheath of FIG. 2.

FIG. 6 is a magnified view of a portion of the expandable sheath of FIG. 2 illustrating expansion of the sheath as a prosthetic device is advanced through the sheath.

FIG. 7 is a magnified, partial cross-sectional view illustrating the constituent layers of the sheath of FIG. 2 disposed on a mandrel.

FIG. 8 is a perspective view of the distal end portion of the expandable sheath in a non-crimped/folded configuration.

FIG. 9 is a perspective view of the distal end portion of the expandable sheath folded around an introducer.

FIG. 10 is an enlarged, cross-section view of the distal end portion folded around the introducer.

FIG. 11 is a perspective view of an example folding device.

FIG. 12 is an exploded view of the folding device of FIG. 11.

FIG. 13 is a cross-section view of the folding device of FIG. 11 in a retracted configuration.

FIG. 14 is a cross-section view of the folding device of FIG. 11 in a contracted configuration.

FIG. 15 is an end view of the folding device of FIG. 11 in a retracted configuration.

FIG. 16 is a perspective view folding device of FIG. 11 in a retracted configuration.

FIG. 17 is an end view of the folding device of FIG. 11 in a partially contracted configuration.

FIG. 18 is an end view of the folding device of FIG. 11 in a contracted configuration.

FIG. 19 is a perspective view of the sheath mounted on a mandrel and received within the folding device.

FIG. 20 is a partial end view of the sheath received within the folding device in a partially contracted configuration.

FIG. 21 is a partial end view of the sheath received within the folding device in a contracted configuration.

FIG. 22 is a perspective view of the folding device, sheath and corresponding mandrels.

FIG. 23 is a perspective view of the mandrels of FIG. 22.

FIG. 24 is a partial end view of the sheath received within an example conventional sheath crimping mechanism.

FIG. 25 is a perspective partial end view of the sheath received within an example conventional sheath crimping mechanism.

FIG. 26 is an end view of the folded and/or compressed sheath.

FIG. 27A-27C are example folded structures of the folded and/or compressed sheath.

FIGS. 28A-28D are schematic representations of the sheath folding process.

FIGS. 29A-29D are schematic representations of the sheath folding process.

FIG. 30 is a conventional sheath crimping mechanism.

FIG. 31 is a perspective view of the distal end portion of the expandable sheath in a non-crimped/folded configuration.

FIG. 32 is a perspective view of the distal end portion of the sheath including a heath-shrink tubing.

FIG. 33 is a perspective view of the distal end of the sheath.

FIG. 34 is a side view of the distal end of the sheath.

FIG. 35 is an end view of the distal end of the sheath.

### DETAILED DESCRIPTION

The expandable introducer sheaths described herein can be used to deliver a prosthetic device through a patient's vasculature to a procedure site within the body. The sheath can be constructed to be highly expandable and collapsible in the radial direction while limiting axial elongation of the sheath and, thereby, undesirable narrowing of the lumen. In one implementation, the expandable sheath includes a braided layer, one or more relatively thin, non-elastic polymeric layers, and an elastic layer. The sheath can resiliently expand from its natural diameter to an expanded diameter as a prosthetic device is advanced through the sheath, and can return to its natural diameter upon passage of the prosthetic device under the influence of the elastic layer. In some implementations, the one or more polymeric layers can engage the braided layer, and can be configured to allow radial expansion of the braided layer while preventing axial elongation of the braided layer, which would otherwise result in elongation and narrowing of the sheath.

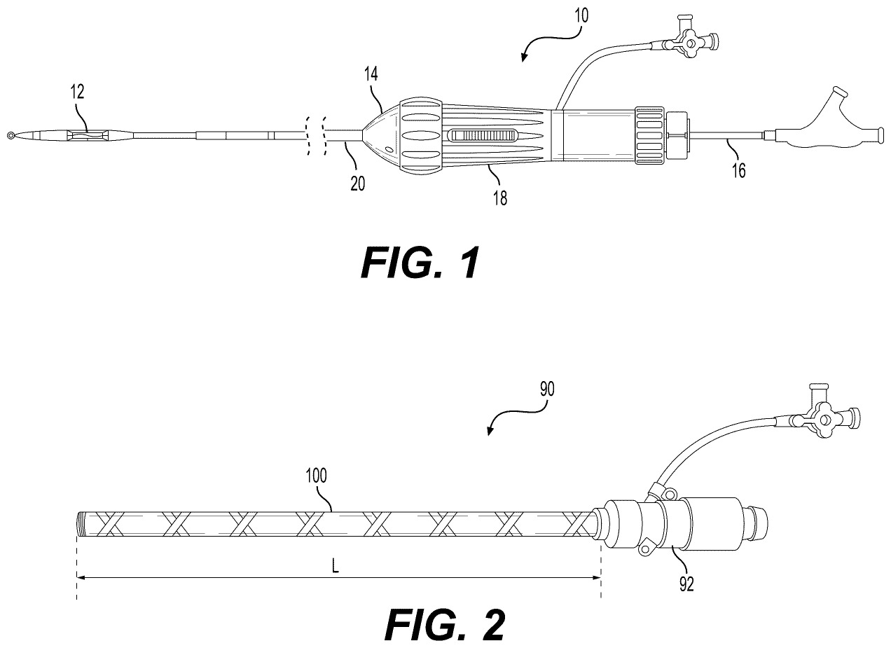

FIG. 1 illustrates a representative delivery apparatus 10 for delivering a medical device, such as a prosthetic heart valve or other prosthetic implant, to a patient. The delivery apparatus 10 is exemplary only, and can be used in combination with any of the expandable sheath implementations described herein. Likewise, the sheaths disclosed herein can be used in combination with any of various known delivery apparatuses. The delivery apparatus 10 illustrated can generally include a steerable guide catheter 14 and a balloon catheter 16 extending through the guide catheter 14. A prosthetic device, such as a prosthetic heart valve 12, can be positioned on the distal end of the balloon catheter 16. The guide catheter 14 and the balloon catheter 16 can be adapted to slide longitudinally relative to each other to facilitate delivery and positioning of a prosthetic heart valve 12 at an implantation site in a patient's body. The guide catheter 14 includes a handle portion 18 and an elongated guide tube or shaft 20 extending from the handle portion 18.

The prosthetic heart valve 12 can be delivered into a patient's body in a radially compressed configuration and radially expanded to a radially expanded configuration at the desired deployment site. In the illustrated example, the prosthetic heart valve 12 is a plastically expandable prosthetic valve that is delivered into the patient's body in a radially compressed configuration on a balloon of the balloon catheter 16 (as shown in FIG. 1) and then radially expanded to a radially expanded configuration at the deployment site by inflating the balloon (or by actuating another type of expansion device of the delivery apparatus). Further details regarding a plastically expandable heart valve that can be implanted using the devices disclosed herein are disclosed in U.S. Publication No. 2012/0123529, which is incorporated herein by reference. In other implementations, the prosthetic heart valve 12 can be a self-expandable heart valve that is restrained in a radially compressed configuration by a sheath or other component of the delivery apparatus and self-expands to a radially expanded configuration when released by the sheath or other component of the delivery apparatus. Further details regarding a self-expandable heart valve that can be implanted using the devices disclosed herein are disclosed in U.S. Publication No. 2012/0239142, which is incorporated herein by reference. In still other implementations, the prosthetic heart valve 12 can be a mechanically expandable heart valve that comprises a plurality of struts connected by hinges or pivot joints and is expandable from a radially compressed configuration to a radially expanded configuration by actuating an expansion mechanism that applies an expansion force to the prosthetic valve. Further details regarding a mechanically expandable heart valve that can be implanted using the devices disclosed herein are disclosed in U.S. Publication No. 2018/0153689, which is incorporated herein by reference. In still other implementations, a prosthetic valve can incorporate two or more of the above-described technologies. For example, a self-expandable heart valve can be used in combination with an expansion device to assist expansion of the prosthetic heart valve.

FIG. 2 illustrates an assembly (which can be referred to as an introducer device or assembly) that can be used to introduce the delivery apparatus 10 and the prosthetic heart valve 12 into a patient's body, according to one implementation. The introducer device 90 can comprise a housing 92 at a proximal end of the device and an expandable sheath 100 extending distally from the housing 92. The housing 92 can function as a handle for the device. The expandable sheath 100 has a central lumen 112 (FIG. 4) to guide passage of the delivery apparatus for the prosthetic heart valve. Generally, during use a distal end of the sheath 100 is passed through the skin of the patient and is inserted into a vessel, such as the femoral artery. The delivery apparatus 10 with its prosthetic heart valve 12 can then be inserted through the housing 92 and the sheath 100, and advanced through the patient's vasculature to the treatment site, where the implant is to be delivered and implanted within the patient. In some implementations, the housing 92 can include a hemostasis valve that forms a seal around the outer surface of the guide catheter 14 once inserted through the housing to prevent leakage of pressurized blood.

In some implementations, the introducer device 90 need not include a housing 92. For example, the sheath 100 can be an integral part of a component of the delivery apparatus 10, such as the guide catheter. For example, the sheath can extend from the handle portion 18 of the guide catheter.

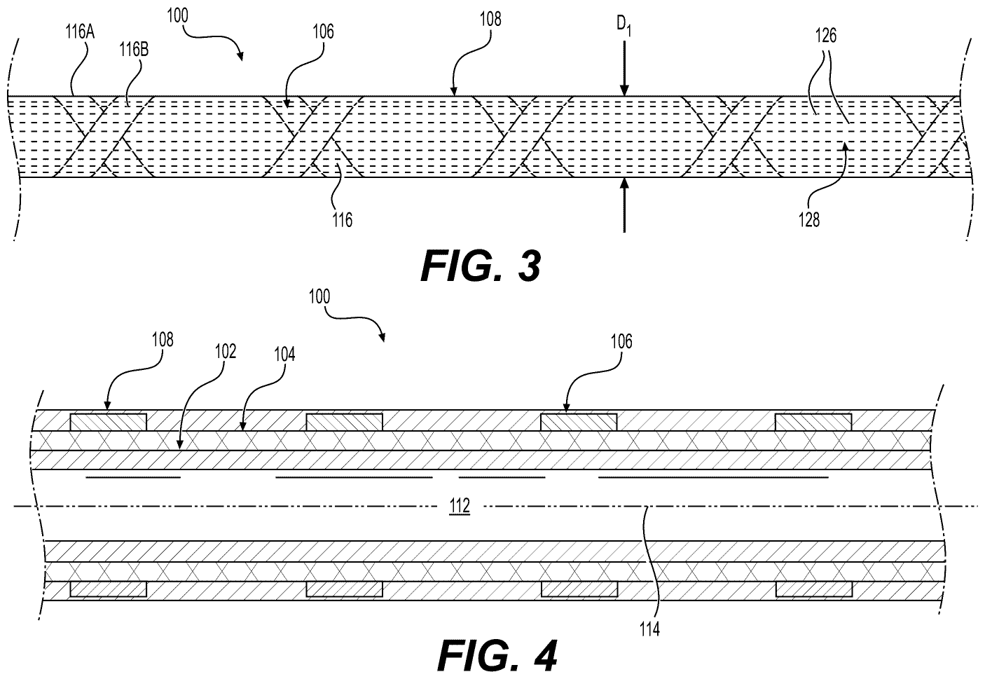

FIG. 3 illustrates the expandable sheath 100 in greater detail. Example expandable introducer sheaths are disclosed in the following U.S. patents and applications, the disclosures of which are herein incorporated by reference in their entirety: U.S. Pat. No. 8,690,936, entitled “Expandable Sheath for Introducing an Endovascular Delivery Device into a Body,” U.S. Pat. No. 8,790,387, entitled “Expandable Sheath for Introducing an Endovascular Delivery Device into a Body,” U.S. Pat. No. 10,639,152, entitled “Expandable Sheath and Methods of Using the Same,” U.S. Pat. No. 10,792,471, entitled “Expandable Sheath,” U.S. patent application Ser. No. 16/407,057, entitled “Expandable Sheath with Elastomeric Cross Sectional Portions,” U.S. Pat. No. 10,327,896, entitled “Expandable Sheath with Elastomeric Cross Sectional Portions,” U.S. Pat. No. 11,273,062, entitled “Expandable Sheath,” Application No. PCT/US2021/019514, entitled “Expandable sheath for introducing an endovascular delivery device in to a body,” Application No. PCT/US2021/031227, entitled “Expandable sheath for introducing an endovascular delivery device into a body,” Application No. PCT/US2021/031275, entitled “Expandable sheath for introducing an endovascular delivery device into a body,” U.S. application Ser. No. 17/113,268, entitled “Expandable Sheath and Method of Using the Same,” Application No. PCT/US2021/058247, entitled “Self-Expanding, Two Component Sheath,” Application No. PCT/US2022/012785, entitled “Expandable Sheath,” U.S. Pat. No. 11,051,939, entitled “Active Introducer Sheath System,” Application No. PCT/US2022/012684, entitled “Introducer with Sheath Tip Expander,” U.S. application Ser. No. 17/078,556, entitled “Advanced Sheath Patterns,” Application No. PCT/US2021/025038, entitled “Low temperature hydrophilic adhesive for use in expandable sheath for introducing an endovascular delivery device into a body,” Application No. PCT/US2021/050006, entitled “Expandable Sheath Including Reversable Bayonet Locking Hub,” U.S. Provisional Application No. 63/280,251, entitled “Expandable Sheath Gasket to Provide Hemostasis,” the disclosures of which are herein incorporated by reference.

With reference to FIG. 3, the sheath 100 can have a natural, unexpanded outer diameter D₁. In some implementations, the expandable sheath 100 can comprise a plurality of coaxial layers extending along at least a portion of the length I. of the sheath (FIG. 2). For example, with reference to FIG. 4, the expandable sheath 100 can include a first layer (also referred to as an inner layer 102), a second layer 104 disposed around and radially outward of the inner layer 102, a third layer 106 disposed around and radially outward of the second layer 104, and a fourth layer 108 (also referred to as an outer layer 108) disposed around and radially outward of the third layer 106. In the illustrated configuration, the inner layer 102 can define the central lumen 112 of the sheath extending along a central axis 114.

Referring to FIG. 3, when the sheath 100 is in an unexpanded state, the inner layer 102 and/or the outer layer 108 can form longitudinally-extending folds or creases such that the surface of the sheath comprises a plurality of ridges (also referred to herein as “folds”, for example, folds/ridges 126). An example sheath is disclosed in U.S. Pat. No. 11,273,062, which is incorporated herein by reference. The folds/ridges 126 can be circumferentially spaced apart from each other by longitudinally-extending valleys 128. When the sheath 100 expands beyond its natural diameter D₁, the folds/ridges 126 and the valleys 128 can level out or be taken up as the surface radially expands and the circumference increases, as further described herein. When the sheath 100 collapses back to its natural diameter, the folds/ridges 126 and valleys 128 can reform.

In some implementations, the inner layer 102 and/or the outer layer 108 can comprise a relatively thin layer of polymeric material. For example, in some implementations the thickness of the inner layer 102 can be from 0.01 mm to 0.5 mm, 0.02 mm to 0.4 mm, or 0.03 mm to 0.25 mm. In some implementations, the thickness of the outer layer 108 can be from 0.01 mm to 0.5 mm, 0.02 mm to 0.4 mm, or 0.03 mm to 0.25 mm.

In some examples, the inner layer 102 and/or the outer layer 108 can optionally comprise a lubricious, low-friction, and/or relatively non-elastic material. In some examples, the inner layer 102 and/or the outer layer 108 can comprise a polymeric material having a modulus of elasticity of 400 MPa or greater. Exemplary materials can include ultra-high-molecular-weight polyethylene (UHMWPE) (for example, Dyneema®), high-molecular-weight polyethylene (HMWPE), or polyether ether ketone (PEEK). With regard to the inner layer 102 in particular, such low coefficient of friction materials can facilitate passage of the prosthetic device through the central lumen 112. Other suitable materials for the inner and outer layers can include polytetrafluoroethylene (PTFE), expanded polytetrafluoroethylene (ePTFE), ethylene tetrafluoroethylene (ETFE), nylon, polyethylene, polyether block amide (for example, Pebax), and/or combinations of any of the above. Some implementations of a sheath 100 can include a lubricious liner on the inner surface of the inner layer 102. Examples of suitable lubricious liners include materials that can further reduce the coefficient of friction of the inner layer 102, such as PTFE, polyethylene, polyvinylidene fluoride, and combinations thereof. Suitable materials for a lubricious liner also include other materials desirably having a coefficient of friction of 0.1 or less.

Additionally, some implementations of the sheath 100 can optionally include an exterior hydrophilic coating on the outer surface of the outer layer 108. Such a hydrophilic coating can facilitate insertion of the sheath 100 into a patient's vessel, reducing potential damage. Examples of suitable hydrophilic coatings include the Harmony™ Advanced Lubricity Coatings and other Advanced Hydrophilic Coatings available from SurModics, Inc., Eden Prairie, MN. DSM medical coatings (available from Koninklijke DSM N.V, Heerlen, the Netherlands), as well as other hydrophilic coatings (for example, PTFE, polyethylene, polyvinylidine fluoride), are also suitable for use with the sheath 100. Such hydrophilic coatings may also optionally be included on the inner surface of the inner layer 102 to reduce friction between the sheath and the delivery system, thereby facilitating use and improving safety. In some implementations, a hydrophobic coating, such as Perylene, may be used on the outer surface of the outer layer 108 or the inner surface of the inner layer 102 in order to reduce friction.

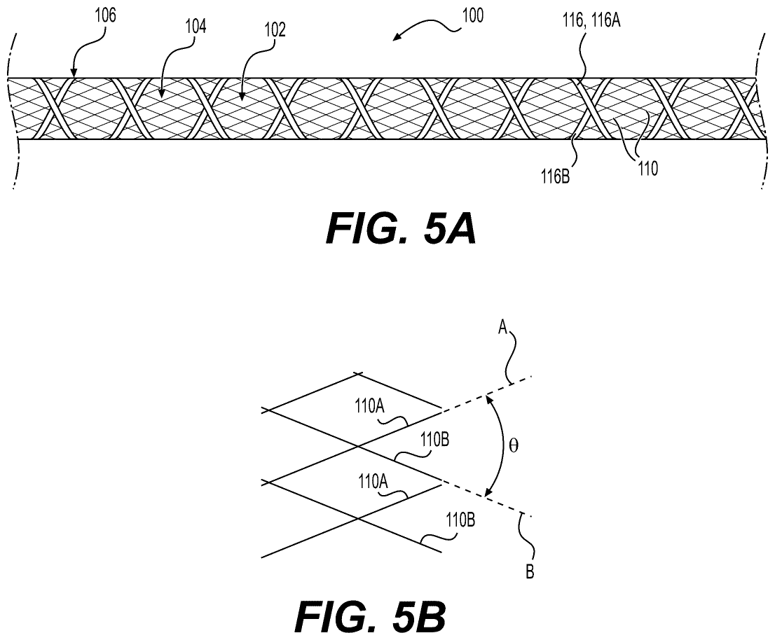

In some implementations, the second layer 104 can optionally be a braided layer (referred to as braided layer 104). In some implementations, the third layer 106 is an elastic layer (referred to as elastic layer 106). FIGS. 5A and 5B illustrate the sheath 100 with the outer layer 108 removed to expose the elastic layer 106. With reference to FIGS. 5A and 5B, the braided layer 104 can comprise a plurality of members or filaments 110 (for example, metallic or synthetic wires or fibers) braided together. The braided layer 104 can have any desired number of filaments 110, which can be oriented and braided together along any suitable number of axes. For example, with reference to FIG. 5B, the filaments 110 can include a first set of filaments 110A oriented parallel to a first axis A, and a second set of filaments 110B oriented parallel to a second axis B. The filaments 110A and 110B can be braided together in a biaxial braid such that filaments 110A oriented along axis A form an angle θ with the filaments 110B oriented along axis B. In some implementations, the angle θ can be from 5° to 70°, 10° to 60°, 10° to 50°, or 10° to 45°. In the illustrated implementation, the angle θ is 45°. In other implementations, the filaments 110 can also be oriented along three axes and braided in a triaxial braid, or oriented along any number of axes and braided in any suitable braid pattern.

The braided layer 104 can optionally extend along substantially the entire length I. of the sheath 100, or alternatively, can extend only along a portion of the length of the sheath. In some implementations, the filaments 110 can be wires made from metal (for example, Nitinol, stainless steel, etc.), or any of various polymers or polymer composite materials, such as carbon fiber. In some implementations, the filaments 110 can be round, and can have a diameter of from 0.01 mm to 0.5 mm, 0.03 mm to 0.4 mm, or 0.05 mm to 0.25 mm. In other implementations, the filaments 110 can have a flat cross-section with dimensions of 0.01 mm×0.01 mm to 0.5 mm×0.5 mm, or 0.05 mm×0.05 mm to 0.25 mm×0.25 mm. In one implementation, filaments 110 having a flat cross-section can have dimensions of 0.1 mm×0.2 mm. However, other geometries and sizes are also suitable for some implementations. If braided wire is used, the braid density can be varied. Some implementations have a braid density of from ten picks per inch to eighty picks per inch, and can include eight wires, sixteen wires, or up to fifty-two wires in various braid patterns. In other implementations, the second layer 104 can be laser cut from a tube, or laser-cut, stamped, punched, etc., from sheet stock and rolled into a tubular configuration. The layer 104 can also be woven or knitted, as desired.

The third layer 106 can optionally be a resilient, elastic layer (also referred to as an elastic material layer). In some implementations, the elastic layer 106 can optionally be configured to apply force to the underlying inner layer 102 and braided layer 104 in a radial direction (for example, toward the central axis 114 of the sheath) when the sheath expands beyond its natural diameter by passage of the delivery apparatus through the sheath. Stated differently, the elastic layer 106 can be configured to apply encircling pressure to the layers of the sheath beneath the elastic layer 106 to counteract expansion of the sheath. The radially inwardly directed force is sufficient to cause the sheath to collapse radially back to its unexpanded state after the delivery apparatus is passed through the sheath.

In the illustrated implementation, the elastic layer 106 can optionally comprise one or more members configured as strands, ribbons, or bands (for example, elastic bands 116) helically wrapped around the braided layer 104. For example, in the illustrated implementation the elastic layer 106 comprises two elastic bands 116A and 116B wrapped around the braided layer with opposite helicity, although the elastic layer may comprise any number of bands depending upon the desired characteristics. The elastic bands 116A and 116B can be made from, for example, any of a variety of natural or synthetic elastomers, including silicone rubber, natural rubber, any of various thermoplastic elastomers, polyurethanes such as polyurethane siloxane copolymers, urethane, plasticized polyvinyl chloride (PVC), styrenic block copolymers, polyolefin elastomers, etc. In some implementations, the elastic layer can comprise an elastomeric material having a modulus of elasticity of 200 MPa or less. In some implementations, the elastic layer 106 can comprise a material exhibiting an elongation to break of 200% or greater, or an elongation to break of 400% or greater. The elastic layer 106 can also take other forms, such as a tubular layer comprising an elastomeric material, a mesh, a shrinkable polymer layer such as a heat-shrink tubing layer, etc. In lieu of, or in addition to, the elastic layer 106, the sheath 100 may also include an elastomeric or heat-shrink tubing layer around the outer layer 108. Examples of such elastomeric layers are disclosed in U.S. Publication No. 2014/0379067, U.S. Publication No. 2016/0296730, and U.S. Publication No. 2018/0008407, which are incorporated herein by reference. In other implementations, the elastic layer 106 can also be radially outward of the polymeric outer layer 108.

In some implementations, one or both of the inner layer 102 and/or the outer layer 108 can optionally be configured to resist axial elongation of the sheath 100 when the sheath expands. More particularly, one or both of the inner layer 102 and/or the outer layer 108 can resist stretching against longitudinal forces caused by friction between a prosthetic device and the inner surface of the sheath such that the length L remains substantially constant as the sheath expands and contracts. As used herein with reference to the length L of the sheath, the term “substantially constant” means that the length L of the sheath increases by not more than 1%, by not more than 5%, by not more than 10%, by not more than 15%, or by not more than 20%. Meanwhile, with reference to FIG. 5B, the filaments 110A and 110B of the braided layer can be allowed to move angularly relative to each other such that the angle θ changes as the sheath expands and contracts. This, in combination with the longitudinal folds/ridges 126 in the inner layer 102 and outer layer 108, can allow the central lumen 112 of the sheath to expand as a prosthetic device is advanced through it.

For example, in some implementations the inner layer 102 and the outer layer 108 can optionally be heat-bonded during the manufacturing process such that the braided layer 104 and the elastic layer 106 are encapsulated between the inner layer 102 and outer layer 108. More specifically, in some implementations the inner layer 102 and the outer layer 108 can optionally be adhered to each other through the spaces between the filaments 110 of the braided layer 104 and/or the spaces between the elastic bands 116. The inner layer 102 and outer layer 108 can optionally also be bonded or adhered together at the proximal and/or distal ends of the sheath. In some implementations, the inner layer 102 and outer layer 108 are not adhered to the filaments 110. This can allow the filaments 110 to move angularly relative to each other, and relative to the inner layer 102 and outer layer 108, allowing the diameter of the braided layer 104, and thereby the diameter of the sheath, to increase or decrease. As the angle θ between the filaments 110A and 110B changes, the length of the braided layer 104 can also change. For example, as the angle θ increases, the braided layer 104 can foreshorten, and as the angle θ decreases, the braided layer 104 can lengthen to the extent permitted by the areas where the inner layer 102 and outer layer 108 are bonded. However, because the braided layer 104 is not adhered to the inner layer 102 and outer layer 108, the change in length of the braided layer that accompanies a change in the angle θ between the filaments 110A and 110B does not result in a significant change in the length L of the sheath.

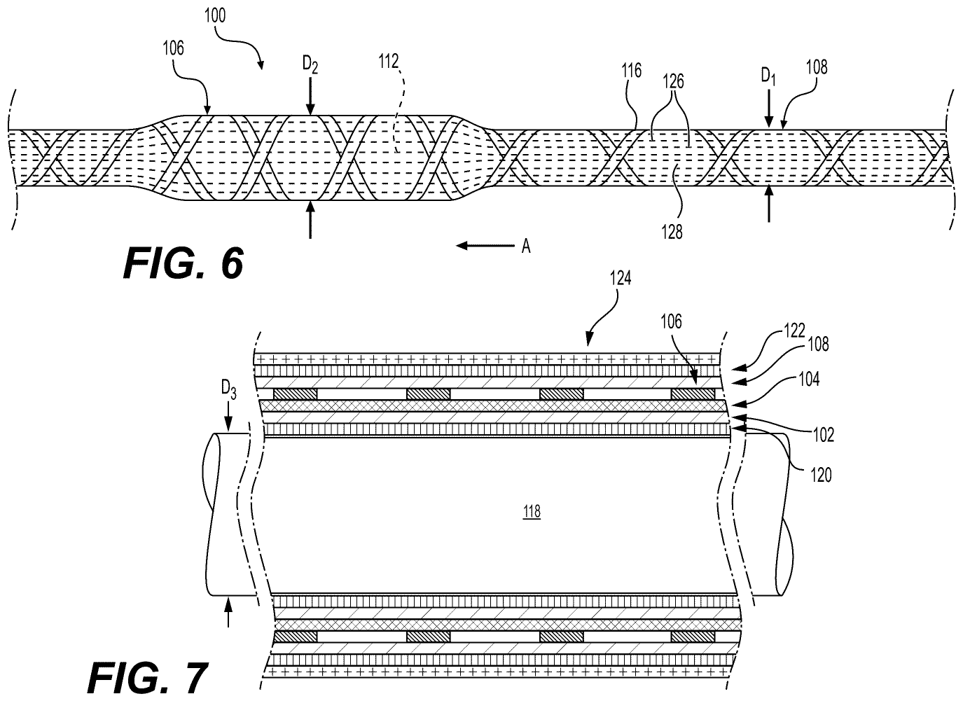

FIG. 6 illustrates radial expansion of the sheath 100 as a prosthetic device (for example, prosthetic heart valve 12) is passed through the sheath in the direction of arrow 132 (for example, distally). As the prosthetic device is advanced through the sheath 100, the sheath can resiliently expand to a second diameter D₂ that corresponds to a size or diameter of the prosthetic device. As the prosthetic device is advanced through the sheath 100, the prosthetic device can apply longitudinal force to the sheath in the direction of motion by virtue of the frictional contact between the prosthetic device and the inner surface of the sheath. However, as noted herein, the inner layer 102 and/or the outer layer 108 can resist axial elongation such that the length I, of the sheath remains constant, or substantially constant. This can reduce or prevent the braided layer 104 from lengthening, and thereby constricting the central lumen 112.

Meanwhile, the angle θ between the filaments 110A and 110B can increase as the sheath expands to the second diameter D₂ to accommodate the prosthetic valve. This can cause the braided layer 104 to foreshorten. However, because the filaments 110 are not engaged or adhered to the inner layer 102 or outer layer 108, the shortening of the braided layer 104 attendant to an increase in the angle θ does not affect the overall length I, of the sheath. Moreover, because of the longitudinally-extending folds/ridges 126 formed in the inner layer 102 and outer layer 108, the inner layer 102 and outer layer 108 can expand to the second diameter D₂ without rupturing, in spite of being relatively thin and relatively non-elastic. In this manner, the sheath 100 can resiliently expand from its natural diameter D₁ to a second diameter D₂ that is larger than the diameter D₁ as a prosthetic device is advanced through the sheath, without lengthening, and without constricting. Thus, the force required to push the prosthetic implant through the sheath is significantly reduced.

Additionally, because of the radial force applied by the elastic layer 106, the radial expansion of the sheath 100 can be localized to the specific portion of the sheath occupied by the prosthetic device. For example, with reference to FIG. 6, as the prosthetic device (for example, prosthetic heart valve 12) moves distally through the sheath 100, the portion of the sheath immediately proximal to the prosthetic device can radially collapse back to the initial diameter D₁ under the influence of the elastic layer 106. The inner layer 102 and outer layer 108 can also buckle as the circumference of the sheath is reduced, causing the folds/ridges 126 and the valleys 128 to reform. This can reduce the size of the sheath required to introduce a prosthetic device of a given size. Additionally, the temporary, localized nature of the expansion can reduce trauma to the blood vessel into which the sheath is inserted, along with the surrounding tissue, because only the portion of the sheath occupied by the prosthetic device expands beyond the sheath's natural diameter and the sheath collapses back to the initial diameter once the device has passed. This limits the amount of tissue that must be stretched in order to introduce the prosthetic device, and the amount of time for which a given portion of the vessel must be dilated.

In addition to the advantages herein, the expandable sheath implementations described herein can provide surprisingly superior performance relative to known introducer sheaths. For example, it is possible to use a sheath configured as described herein to deliver a prosthetic device having a diameter that is two times larger, 2.5 times larger, or even three times larger than the natural outer diameter of the sheath. For example, in one implementation a crimped prosthetic heart valve having a diameter of 7.2 mm was successfully advanced through a sheath configured as described herein and having a natural outer diameter of 3.7 mm. As the prosthetic valve was advanced through the sheath, the outer diameter of the portion of the sheath occupied by the prosthetic valve increased to 8 mm. In other words, it was possible to advance a prosthetic device having a diameter more than two times the outer diameter of the sheath through the sheath, during which the outer diameter of the sheath resiliently increased by 216%. In another example, a sheath with an initial or natural outer diameter of 4.5 mm to 5 mm can be configured to expand to an outer diameter of 8 mm to 9 mm.

In alternative implementations, the sheath 100 may optionally include the layer 102 without the outer layer 108, or the outer layer 108 without the layer 102, depending upon the particular characteristics desired.

Turning now to methods of making expandable sheaths, FIG. 7 illustrates the layers (for example, inner layer 102 through outer layer 108) of the expandable sheath 100 disposed on a cylindrical mandrel 118, according to one implementation. In some implementations, the mandrel 118 can have a diameter D₃ that is greater than the desired natural outer diameter D₁ of the finished sheath. For example, in some implementations a ratio of the diameter D₃ of the mandrel to the outer diameter D₁ of the sheath can be 1.5:1, 2:1, 2.5:1, 3:1, or greater. In some implementations, the diameter D₃ of the mandrel can be equal to the expanded diameter D₂ of the sheath. In other words, the diameter D₃ of the mandrel can be the same, or nearly the same, as the desired expanded diameter D₂ of the sheath when a prosthetic device is being advanced through the sheath. Thus, in some implementations a ratio of the expanded outer diameter D₂ of the expanded sheath to the collapsed outer diameter D₁ of the unexpanded sheath can be 1.5:1, 2:1, 2.5:1, 3:1, or greater.

With reference to FIG. 7, the expandable sheath 100 can optionally be made by wrapping or situating an ePTFE layer 120 around the mandrel 118, followed by the first polymeric layer 102. In some implementations, the ePTFE layer can aid in removing the sheath 100 from the mandrel 118 upon completion of the fabrication process. The first polymeric layer 102 may be in the form of a pre-fabricated sheet that is applied by being wrapped around the mandrel 118, or may be applied to the mandrel by dip-coating, electro-spinning, etc. The braided layer 104 can be situated around the inner layer 102, followed by the elastic layer 106. In implementations in which the elastic layer 106 comprises one or more elastic bands 116, the elastic bands 116 can be helically wrapped around the braided layer 104. In other implementations, the elastic layer 106 may be dip-coated, electro-spun, etc. The polymeric outer layer 108 can then be wrapped, situated, or applied around the elastic layer 106, followed by another layer 122 of ePTFE and one or more layers 124 of heat-shrink tubing or heat-shrink tape.

In some implementations, the elastic bands 116 can be applied to the braided layer 104 in a stretched, taut, or extended condition. For example, in some implementations the elastic bands 116 can be applied to the braided layer 104 stretched to a length that is twice their natural, relaxed length. This will cause the completed sheath to radially collapse under the influence of the elastic layer when removed from the mandrel, which can cause corresponding relaxation of the elastic layer, as described herein. In other implementations, the layer 102 and the braided layer 104 can be removed from the mandrel, the elastic layer 106 can be applied in a relaxed state or moderately stretched state, and then the assembly can be placed back on the mandrel such that the elastic layer is radially expanded and stretched to a taut condition prior to application of the outer layer 108.

The assembly can then be heated to a sufficiently high temperature that the heat-shrink layer 124 shrinks and compresses the layers (for example, inner layer 102 to outer layer 108) together. In some implementations, the assembly can be heated to a sufficiently high temperature such that the polymeric inner layer 102 and outer layer 108 become soft and tacky, and bond to each other in the open spaces between the braided layer 104 and the elastic layer 106 and encapsulate the braided layer and the elastic layer. In other implementations, the inner layer 102 and outer layer 108 can be reflowed or melted such that they flow around and through the braided layer 104 and the elastic layer 106. In an exemplary implementation, the assembly can be heated at 150° C. for 20-30 minutes.

After heating, the sheath 100 can be removed from the mandrel 118, and the heat-shrink layer 124 and the ePTFE layers 120 and 122 can be removed. Upon being removed from the mandrel 118, the sheath 100 can at least partially radially collapse to the natural design diameter D₁ under the influence of the elastic layer 106. In some implementations, the sheath can be radially collapsed to the design diameter with the optional aid of a crimping mechanism. The attendant reduction in circumference can buckle the filaments 110 along with the inner layer 102 and outer layer 108 to form the longitudinally-extending folds/ridges 126.

In some implementations, a layer of PTFE can optionally be interposed between the ePTFE layer 120 and the inner layer 102, and/or between the outer layer 108 and the ePTFE layer 122, in order to facilitate separation of the polymeric layers, inner layer 102 and outer layer 108, from the respective ePTFE layers 120 and 122. In some implementations, one of the inner layer 102 or the outer layer 108 may be omitted, as described herein.

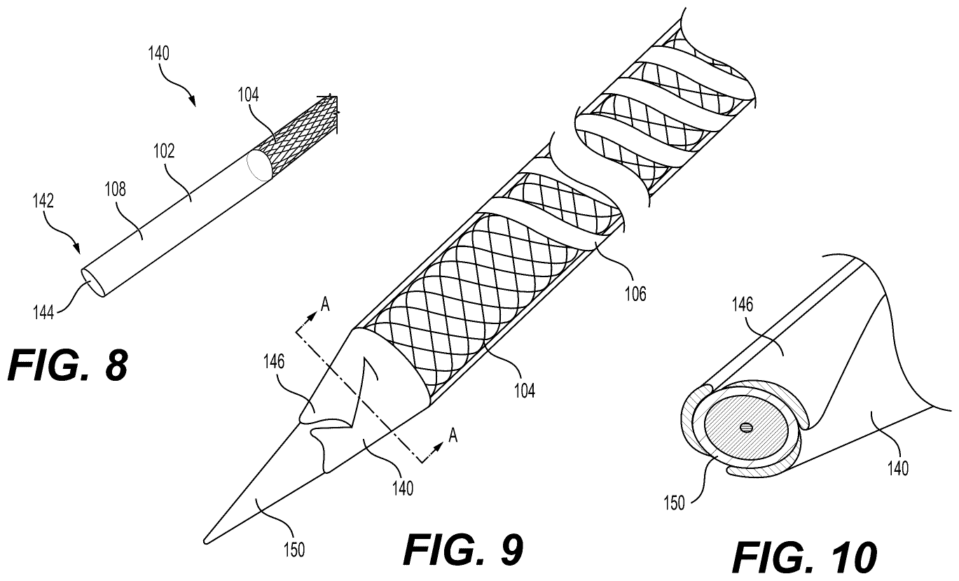

As illustrated in FIGS. 8-10 and FIGS. 31-35, the sheath 100 includes a distal end portion 140 provided at the distal end 142 of the sheath 100. The distal end portion 140 defines the opening 144 at the distal end 142 of the central lumen 112 of the sheath 100. In some implementations, as described herein, the distal end portion 140 can define a portion of the sheath 100 having a reduced diameter compared to the elongated body portion of the sheath 100. In some implementations, the distal end portion 140 of the sheath 100 is constructed as an extension of the inner layer 102 and/or the outer layer 108 extending beyond the distal end of the braided layer 104. For example, in some implementations, the distal end portion 140 includes an extension of the inner layer 102 and/or outer layer 108 of the sheath 100, with or without one more additional layers. The distal end portion 140 can extend distally beyond the distal end of the braided layer 104 and elastic layer 106. In some implementations, the braided layer 104 may extend distally beyond the elastic layer 106, and the distal end portion 140 extends distally beyond both the braided layer 104 and elastic layer 106, as shown in FIGS. 8-10 and FIGS. 31-35. In some implementations, the distal end portion is 140 is formed separate from the sheath 100 and added/coupled to the distal end of 142 the sheath 100. For example, the distal end portion 140 can be formed from a separate, multilayer tubing that is heat bonded to the remainder of the sheath 100 (for example, prior to any tip folding steps).

In some implementations, the distal end portion 140 is formed of one or more layers of a similar or the same material used to form the inner layer 102 and/or outer layer 108 of the sheath 100. For example, in some implementations, the distal end portion 140 can include a single layer of material. In some implementations, the distal end portion 140 can include multiple layers of material. For example, the distal end portion 140 can include from 1 to 8 layers of material (including 1, 2, 3, 4, 5, 6, 7, and 8 layers of material). In some implementations, the distal end portion 140 includes greater than 8 layers of material. In some implementations, the distal end portion 140 comprises multiple layers of a Dyneema® material.

As described herein, in some implementations, the folded portions 146 and/or crimping can be including along the distal end portion 140 of the sheath 100 to adjust the radial and/or longitudinal geometry of the sheath 100. The distal end portion 140 can expand and/or unfold during passage of the delivery system and/or prosthetic heart valve 12 through the distal end portion 140/distal opening of the sheath 100. By reducing the diameter of the distal end portion 140 of the sheath 100, the push force needed to advance the sheath 100 through the patient's blood vessel is reduced, thereby reducing the risk of trauma to the blood vessel and damage to the sheath 100 and prosthetic heart valve 12.

In some implementations, as shown in FIGS. 9 and 10, the distal end portion 140 can be optionally folded and/or collapsed to provide a tapered geometry. In some examples, the distal end portion 140 is tapered to the diameter of an introducer and/or vessel dilator received within the central lumen 112 of the sheath 100 and used during insertion of the sheath 100 and/or expansion of the vessel wall. For example, as illustrated in FIGS. 9 and 10, the tapered shape of the distal end portion 140 is sized and shaped to correspond to the tapered structure of the distal end of the introducer 150. FIG. 10 shows a cross-section of the distal end portion 140 folded around the introducer 150 (in the crimped and/or collapsed configuration) taken along section line A-A in FIG. 9. The distal end portion 140 may have a smaller folded/collapsed diameter than the more proximal portions of the sheath 100, giving it a tapered configuration that smooths the transition between the introducer/dilator and the sheath 100, ensuring that the sheath 100 does not get lodged against the tissue during insertion into the patient. Exemplary folding methods and configurations are described in U.S. patent application Ser. Nos. 14/880,109 and 14/880,111, each of which are hereby incorporated by reference in their entireties. Scoring can optionally be used as an alternative, or in addition to folding of the distal end portion. Both scoring and folding of the distal end portion 140 allow for the expansion of the distal end portion upon the passage of the delivery system, and ease the retraction of the delivery system back into the sheath 100 once the procedure is complete.

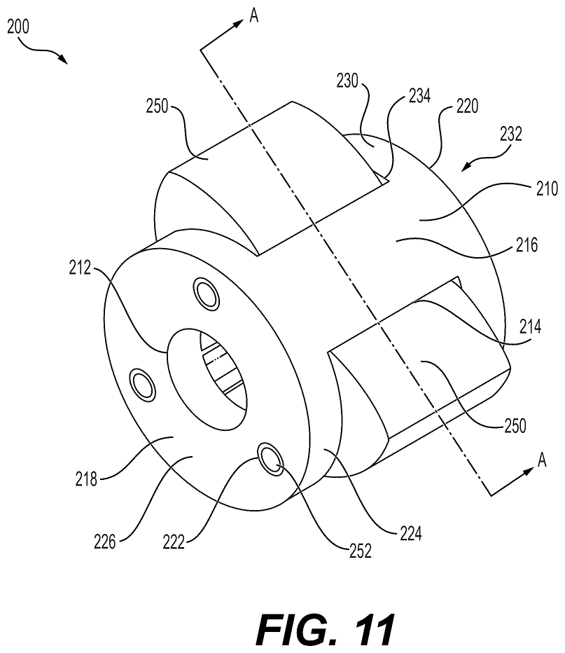

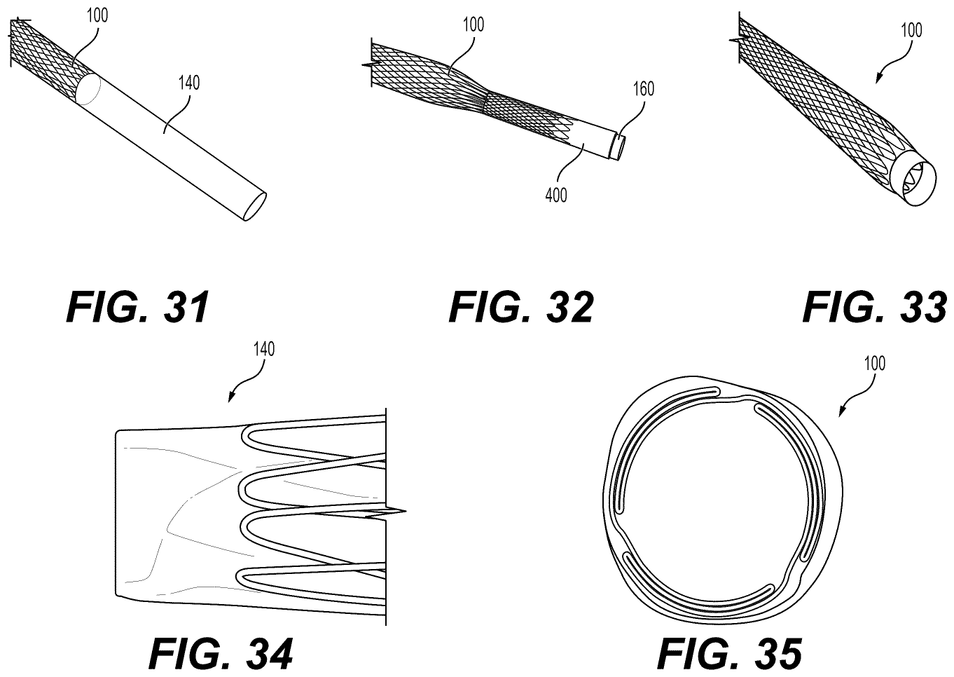

In some implementations, described herein in reference to the sheath 100 shown in FIGS. 31-35, and the folding device shown in FIGS. 11-25, the distal end portion 140 of the sheath 100 can include one or more folded portions 146 extending axially along the shath 100 that adjust the radial and/or longitudinal geometry of the sheath 100. As illustrated in FIGS. 31-35, in the folded configuration, the distal end portion 140 does not include a tapered distal end 142 but rather has a generally constant diameter along the distal end portion 140 of the sheath 100. This provides for a distal end portion 140 of the sheath 100 having a reduced diameter, reducing the risk of damage to the blood vessel during positioning of the sheath 100 at the treatment site by reducing push forces necessary to advance the sheath 100 through the blood vessel and minimizing the size of the needed vessel opening and expansion. The smaller folded/collapsed diameter can be a result of multiple folds (for example, 1, 2, 3, 4, 5, 6, 7, 8 folds, or greater than 8 folds) positioned circumferentially (evenly or unevenly spaced) around the distal end portion 140. For example, as described herein, a circumferential segment of the distal end portion 140 can be brought together and then laid against the adjacent outer surface of the distal end portion 140 to create an overlapping fold. In the collapsed configuration, the overlapping portions of the fold extend longitudinally along the distal end portion 140.

Crimping and/or folding of the example expandable sheath 100 described herein can be performed in a variety of ways. For example, in some implementations, the sheath 100 can be crimped using a conventional short crimper several times longitudinally along the length of the sheath 100. In other implementations, the sheath 100 may be collapsed to a specified crimped diameter in one or a series of stages in which the sheath 100 is wrapped in heat-shrink tubing and collapsed under heating. For example, a first heat shrink tube can be applied to the outer surface of the sheath 100, the sheath 100 can be compressed to an intermediate diameter by shrinking the first heat shrink tube (via heat), the first heat shrink tube can be removed, a second heat shrink tube can be applied to the outer surface of the sheath 100, the second heat shrink tube can be compressed via heat to a diameter smaller than the intermediate diameter, and the second heat shrink tube can be removed. This can go on for as many rounds as necessary to achieve the desired crimped sheath diameter.

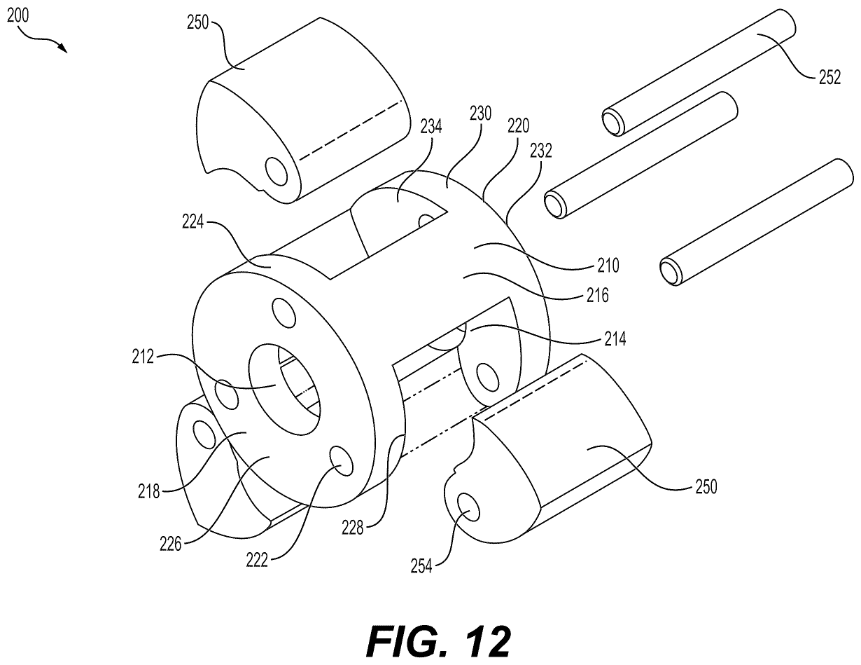

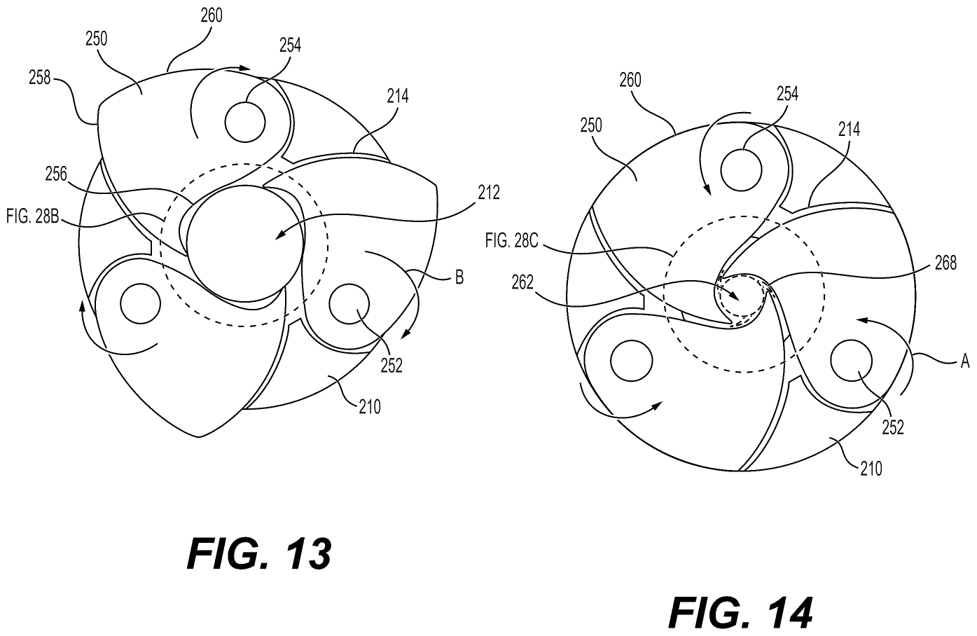

In another example, a folding device, such as the one shown in FIGS. 11-25, can be used for folding and/or crimping the example sheath 100 described herein. FIG. 11 shows a perspective view of the folding device 200 and FIG. 12 shows an exploded perspective view of the folding device 200. The folding device 200 includes a housing 210 with a central opening 212 extending therethrough. The central opening 212 is sized and configured to receive an unfolded portion (for example, distal end portion 140) of the sheath 100. The folding device 200 includes a plurality of jaws 250 rotatably coupled to a housing 210 and movable between a first position (for example, retracted position, shown in FIGS. 13, 15, and 16) and a second position (for example, contracted position, shown in FIGS. 14, 17, and 18). As will be described in more detail herein, with the jaws 250 in the first position (retracted position), the distal end portion 140 of the sheath 100 can be inserted into the central opening 212. The jaws 250 are then moved to/toward the second position (contracted position) and the portion of the sheath 100 within the housing 210 (for example, distal end portion 140) is collapsed and/or folded into the configuration illustrated in FIGS. 32-35.

As illustrated in FIGS. 11-12, the housing 210 includes a plurality of channels 214 extending through the side wall 216 of the housing 210. The channels 214 are positioned circumferentially around the housing 210. As illustrated in FIGS. 11-12, the channels 214 are positioned symmetrically around the circumference of the housing 210. Each of the plurality of jaws 250 is rotatable within a corresponding one of the plurality of channels 214. The jaws 250 are rotatably coupled to the housing 210 at a corresponding axel 252 extending between a proximal end 218 and distal end 220 of the housing 210, where the axel 252 extends through a jaw axel bore 254 provided in each of the jaws 250.

As illustrated in FIG. 11, each of the axels 252 extend through and/or into the housing 210 at a corresponding housing axel bore 222. The housing 210 includes a proximal end portion 224 extending between a proximal end surface 226 of the housing 210 and a proximal end surface of each 228 of the plurality of channels 214. The housing 210 also includes a distal end portion 230 extending between a distal end surface 232 of the housing 210 and a distal end surface 324 of each of the plurality of channels 214. Each of the housing axel bores 222 extend through the corresponding portions of the proximal end portion 224 and distal end portion 230 of the housing 210.

FIGS. 13 and 14 show cross-section views of the folding device 200 taken in a plane perpendicular to the longitudinal axis of the housing 210 as illustrated by section line A-A in FIG. 11. As described herein, the jaws 250 are movable between the first position (retracted position, shown in FIG. 13) and the second position (contracted position, shown in FIG. 14) for folding/crimping and/or collapsing a portion of the sheath 100 received within the central opening 212. As illustrated in FIGS. 13 and 14, each of the jaws 250 includes a first inner surface 256 (leading surface), a second inner surface 258 (trailing surface), and an outer surface 260, where the second inner surface 258 is located between the first inner surface 256 and the outer surface 260. In some implementations, the first inner surface 256 (leading surface) defines a concave surface and the second inner surface 258 (trailing surface) defines a convex surface. In some implementations, the curvature of the outer surface 260 corresponds with an outer curvature of the housing 210. In this implementation, when the jaws 250 are in the first position (retracted position shown in FIG. 13), the outer surface 260 is aligned with the outer surface of the housing 210.

As shown in FIG. 13, when the jaws 250 are in the first position (retracted position), the first inner surface 256 (leading surface) is positioned proximate, for example, adjacent and/or aligned with, but exterior to the central opening 212 extending through the housing 210. As shown in FIG. 14, as the jaws 250 move from the first position (retracted position) to the second position (contracted position), the first inner surface 256 and the second inner surface 258 extend into the central opening 212 of the housing 210.

The jaws 250 can rotate on axels 252 between the first and second positions in a coordinated matter. For example, each of the jaws 250 rotate in the direction A (see FIG. 14) to/toward the first position (retracted position) at the same time and speed. Likewise, each of the jaws 250 can rotate in direction B (see FIG. 13) to/toward the second position (contracted position) configuration at the same time and speed. In some implementations, the jaws 250 can be separately/independently rotated between the first and second position. In some implementations, as the jaws 250 rotate from the first position (retracted position) to the second position (contracted position), each of the jaws 250 is displaced and/or rotated inward forming an opening 262 between adjacent jaws configured to capture portions of the sheath 100 there-between. As a result, the portion of the sheath 100 captured with in the opening 262 is folded and/or collapsed, forming folded portions of the sheath 100 disposed around its circumference in an orderly and symmetrical manner. In some implementations, the folded portions of the sheath 100 extend longitudinally along and circumferentially around the sheath 100, as shown in FIG. 9. As shown in FIG. 14, the opening 262 can define a star-shaped and/or crescent-shaped opening 268, such that the arms of the crescent/star-shaped opening correspond to the folded portions of the sheath 100.

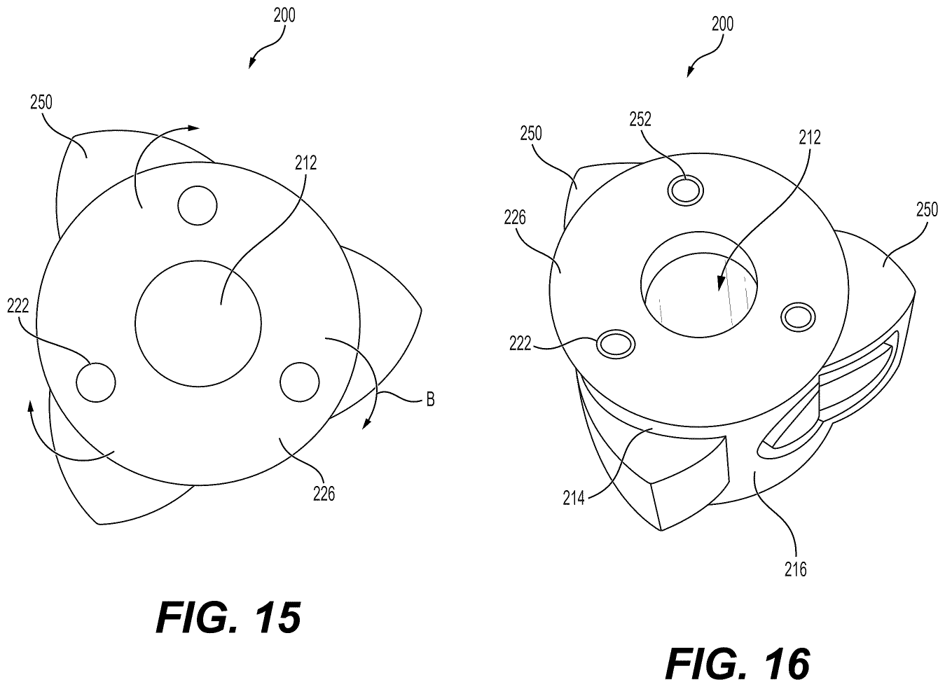

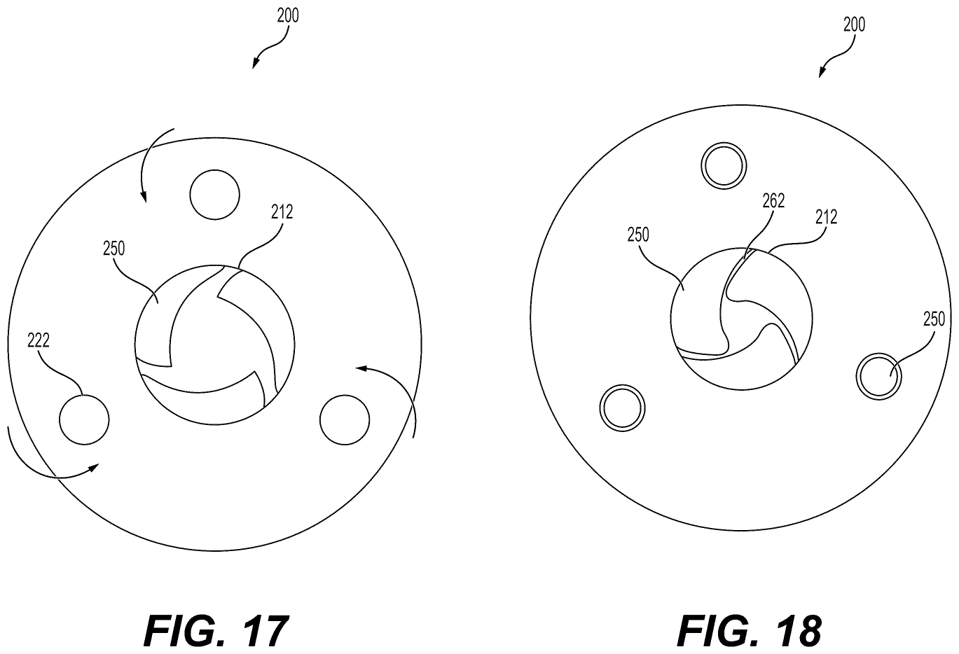

FIG. 15 is an end view of the folding device 200 in the first position (retracted position), and FIG. 16 is a perspective end view of the folding device 200 in a first position (retracted position). As shown in FIGS. 15 and 16, with the jaws 250 in the first position (retracted position), the central opening 212 of the housing 210 is unobstructed, i.e., the jaws 250 do not extend into the central opening 212. FIG. 17 is an end view of the folding device 200 in a partially contracted position, and FIG. 18 is an end view of the folding device 200 in a fully contracted position. As shown in FIGS. 17 and 18, as the jaws 250 move from the first position (retracted position) to and/or toward the second position (contracted position), at least a portion of each of the jaws 250 extend into the central opening 212 of the housing 210.

The method of using the folding device 200 to fold, crimp and/or collapse a portion of the sheath 100 is described herein. In some implementations, the folding device 200 is used to form symmetrical folds around the distal end portion 140 of the sheath 100. The folding device 200 can optionally be used to form symmetric folds around the elongated body portion of the sheath 100, that is, the portion of the sheath 100 extending between the distal end portion 140 and the proximal end of the sheath 100.

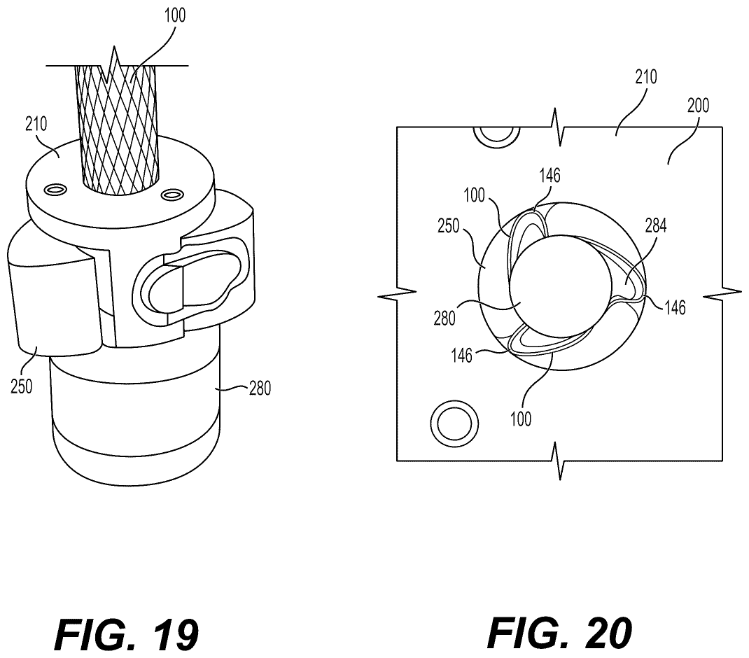

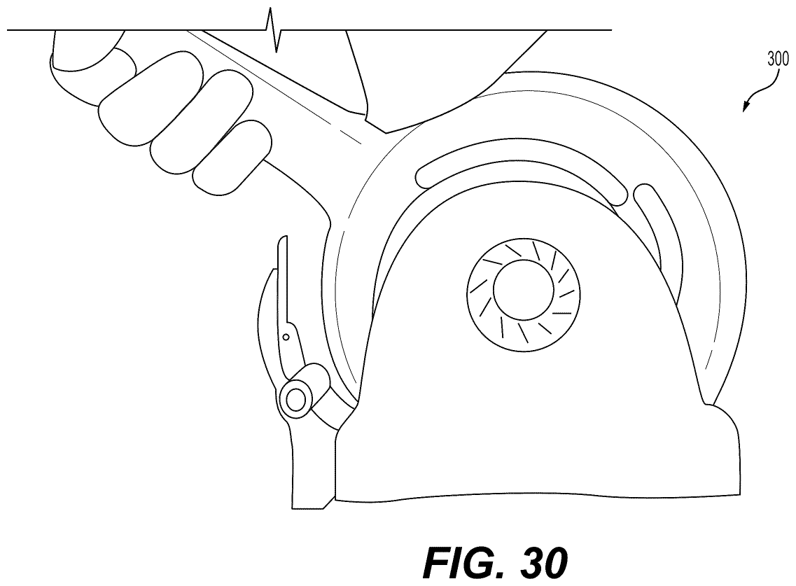

The present example describes the method of folding the distal end portion 140 of an example sheath 100 as illustrated in FIG. 30. In some examples, as provided in FIG. 30, the distal end portion 140 of the sheath 100 has a diameter corresponding to the diameter of the elongated body portion of the sheath 100. As illustrated in FIG. 19, during the folding/crimping procedure, the jaws 250 are configured in the first position (retracted position) and the uncompressed sheath 100 (for example, the uncompressed distal end portion 140 of the sheath 100) is advanced into central opening 212 of the folding device 200 and into the space between the retracted jaws 250. In some implementations, the sheath 100 extends through the central opening 212 and beyond the distal end surface 232 of the housing 210. In other implementations, the sheath 100 extends into the central opening 212, but not beyond the distal end surface 232 of the housing 210.

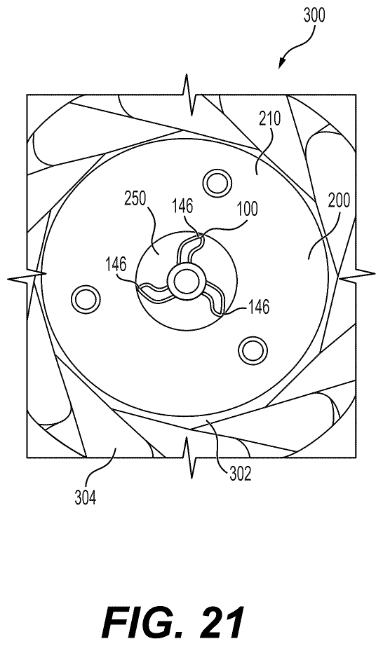

The jaws 250 are then rotated radially inward (for example, direction A) from the first position (retracted position) to/toward the second position (contracted position) such that at least a portion of the jaws 250 are displaced into the central opening 212 of the housing 210. As illustrated in FIGS. 20 and 21, as the jaws 250 move from the first position toward/to the second position, portions of the uncompressed sheath 100 are captured between adjacent jaws 250 thereby creating the folded portions 146 of the sheath 100 and compressing the sheath 100 (for example, folding and/or compressing the distal end portion 140 of the sheath 100). FIG. 20 shows the jaws 250 advanced partially toward the second position (contracted position) and FIG. 21 shows the jaws 250 in the second position (contracted position).

In some implementations, the jaws 250 are moved from the first position toward the second position in a coordinated manner thereby folding the sheath 100 in an orderly and symmetrical manner. In some implementations, the jaws 250 are separately/independently rotated between the first and second positions. As such, the folded portions 146 of the sheath 100 can be formed separately, for example, sequentially, around the circumference of the sheath 100.

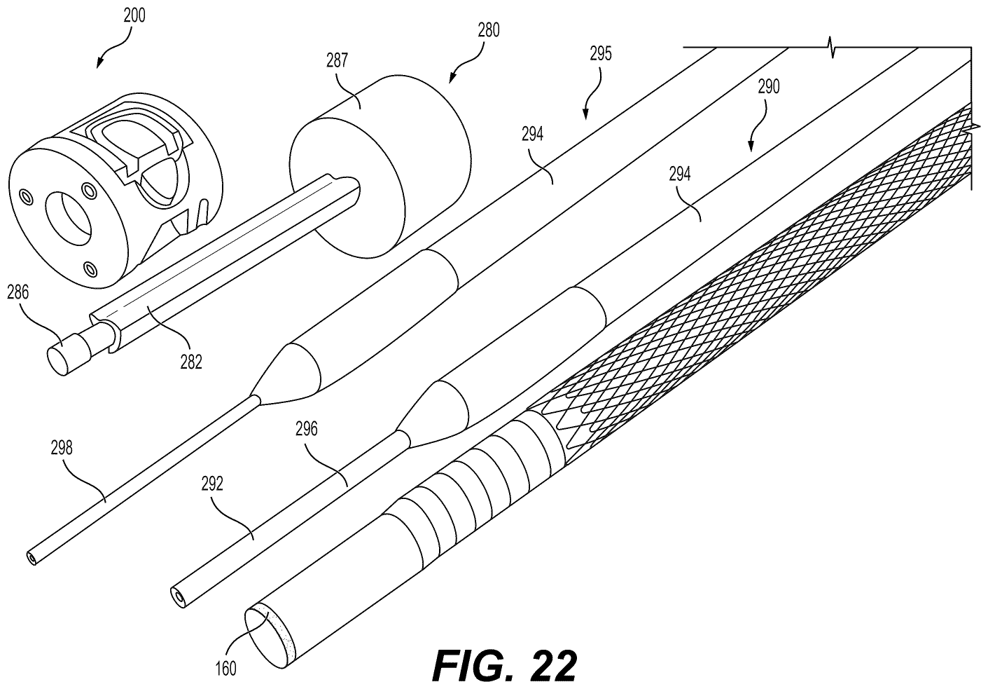

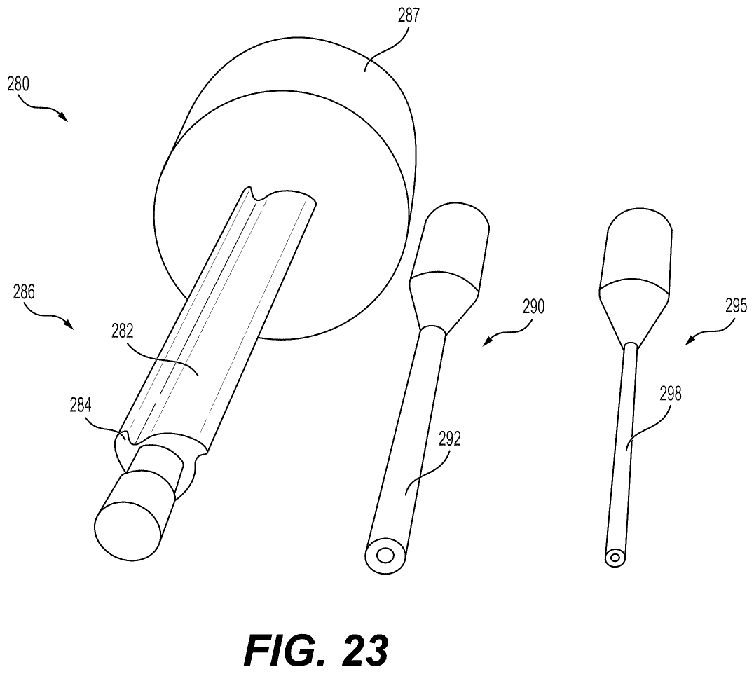

In some implementations, as illustrated in FIG. 20, a mandrel 280 is used to support and/or assist in folding, crimping or otherwise compressing the sheath 100 during an initial folding step. The mandrel 280 provides a support structure for the sheath 100 during the initial folding step. FIGS. 22 and 23 show end perspective views of an example mandrel 280. The mandrel 280 includes a body portion 286 extending from a larger diameter base 287. The mandrel 280 is sized and configured to be received within the central lumen 112 of the uncompressed sheath 100 and the central opening 212 of the housing 210. The mandrel 280 is sized and configured to be received within the central opening 212 of the housing 210 when the jaws 250 are in the first position and when they are at least partially advanced toward the second position. In some implementations, as illustrated in FIGS. 20 and 22-23, at least a portion 282 of the mandrel 280 has a star-shaped cross section. In some implementations, the portion 282 of the mandrel 280 including the star-shaped cross section includes crescent-shaped wings 284 extending at an angle radially outward from the body portion 286 of the mandrel 280. As shown in FIG. 20, with the jaws 250 fully or partially positioned in the second position (contracted position), the crescent-shaped wings 284 define a shape complementary to the crescent/star-shaped opening 262 (FIG. 14) defined by the inner surfaces of the jaws 250 during folding/compression of the sheath 100.

As illustrated in FIG. 20, during the initial folding step of the folding/compression procedure, the body portion 286 and the star-shaped portion 282 of the mandrel 280 are inserted within the central lumen 112 of the uncompressed sheath 100. For example, in some implementations, the mandrel 280 is inserted within the central lumen 112 of the sheath 100 after the distal end portion 140 of the uncompressed sheath 100 is inserted into the central opening 212 folding device 200. As such, the distal end portion 140 of the sheath 100 is supported by the body portion 286/star-shaped portion 282 of the mandrel 280 during the initial folding step.

As illustrated in FIG. 20, as the jaws 250 are moved from the first position (retracted position) toward the second position (contracted position), the jaws 250 are rotated radially inward (for example, direction A) and portions of the uncompressed sheath 100 are captured between adjacent jaws 250 and compressed against the mandrel wings 284. As a result, the folded portions 146 of the sheath 100 are thereby formed along the portion of the sheath 100 received within the folding device 200/housing 219.

In some implementations, a second mandrel 290 is used to support and/or assist in further folding/compressing the sheath 100 during an additional folding step illustrated, for example, in FIG. 21. The second mandrel 290 is sized and configured to be received with the central lumen of the initially compressed/folded sheath 100 (and the central opening 212 of the folding device 200). As shown in FIGS. 22 and 23, comparing the mandrel 280 with the second mandrel 290, the diameter of the portion of the second mandrel 290 that supports the folded portion of the sheath 100 is less than the diameter of the corresponding portion of the mandrel 280. Additionally, the second mandrel 290 has a circular-shaped cross section 292, in contrast to the star-shaped cross section of the mandrel 280. As illustrated in FIGS. 22 and 23, the second mandrel 290 has a large diameter portion 294 with a diameter corresponding to the uncompressed diameter of the sheath 100. As a result, when the second mandrel 290 is received within the central lumen 112 of the sheath 100, the distal end portion 140 of the sheath 100 is received along the smaller diameter portion 296, and elongated body portion of the sheath 100 is received along the large diameter portion 294. For example, in some implementation, the smaller diameter portion 296 is received within the distal end portion 140 of the sheath 100 during the additional folding/compression step.

In some implementations, as provided in FIGS. 22 and 23, the second mandrel 290 includes a tapering segment extending between the large diameter portion 294 and the smaller diameter portion 296. Like the large diameter portion 294 and the smaller diameter portion 296, the tapering segment has a circular-shaped cross section 292.

During the additional folding/compression step, after the sheath 100 is initially folded using the star-shaped mandrel 280, the star-shaped mandrel 280 is removed from the sheath 100 and the second mandrel 290 is advanced into the central lumen of the sheath 100, as illustrated in FIG. 21. In some implementations, the jaws 250 are at least partially rotated toward the first position (retracted position) in direction B, before the second mandrel 290 is advanced into the sheath 100. In other implementations, the jaws 250 remain in the second position (contracted position) when the star-shaped mandrel 280 is removed and the second mandrel 290 inserted.

With the second mandrel 290 positioned within the sheath 100, the jaws 250 are then rotated in direction A toward the second position (contracted position) by rotating each of the jaws 250 radially inward as illustrated in FIG. 21. As a result, the folded portions of the sheath 100 are further compressed and/or crimped against the outer surface of the second mandrel 290.

The second mandrel 290 is then withdrawn from the folded/compressed portion of the sheath 100 and the central opening 212 of the folding device 200. For example, when used to fold the distal end portion 140, the second mandrel 290 is withdrawn from the folded/compressed portion of the sheath 100 as well as the central opening 212 of the folding device 200.

In some implementations, the folded/compressed portion of the sheath 100 is also removed from the central opening 212 of the folding device 200. For example, the sheath 100 is removed from the central opening 212 of the folding device 200 and the second mandrel 290 then withdrawn from the central opening 212 (for example, distal end portion 140) of the sheath 100.

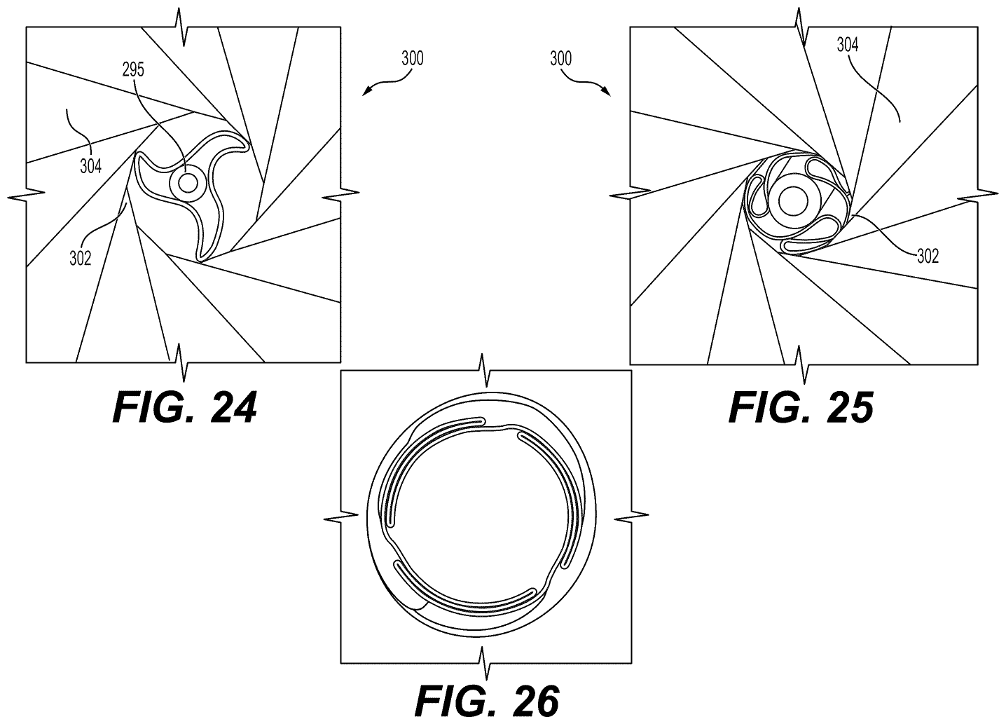

In some implementations, the sheath 100 is optionally further compressed such that the folded portions 146 of the sheath 100 are compressed and/or crimped against the inner and outer surfaces of the sheath 100. As illustrated in FIG. 24, a mandrel is inserted into the central lumen of the folded/compressed sheath 100. In some implementations, the second mandrel 290 is used. In other implementations, a third mandrel 295 is advanced within the central lumen of the sheath 100. The third mandrel 295 has a smaller diameter portion 298 having a diameter less than the diameter of the smaller diameter portion 296 of the second mandrel 290. In some implementations, the second mandrel 290 and or third mandrel 295 can be from about 2 millimeters to about 4 millimeters in diameter. In some implementations, the diameter of the smaller diameter portion 296 and/or smaller diameter portion 298 ranges from about 2 millimeters to about 4 millimeters, including about 2.2 millimeters, about 2.4 millimeters, about 2.6 millimeters, about 2.8 millimeters, about 3.0 millimeters, about 3.2 millimeters, about 3.4 millimeters, about 3.6 millimeters, about 3.8 millimeters and about 4.0 millimeters. In some implementations, the third mandrel 295 is used throughout the folding/crimping process to internally support the layers of the sheath 100 during each of the compression/folding/pleating steps. Utilizing the second mandrel 290 and/or third mandrel 295 in the final folding step supports the internal circular shape of the compressed distal end portion 140 of the sheath 100.

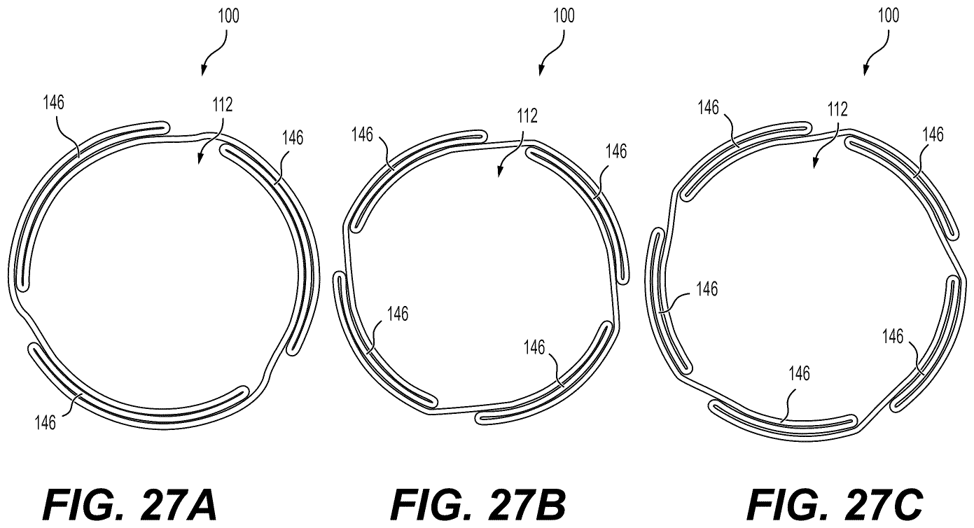

As illustrated in FIG. 25, with sheath 100 is mounted on the second mandrel 290 or third mandrel 295. The sheath 100 is then further compressed by an inwardly directed radial force provided on the exterior of the folded/compressed portions 146 of the sheath 100. Compressing the folded/compressed portion 146 of the sheath 100, for example, the folded/compressed distal end portion 140, includes compressing the folded portions 146 toward and/or against the outer surface of the second mandrel 290 or third mandrel 295. Compression of the folded portions 146 of the sheath 100 against/toward the outer surface of the second mandrel 290 or third mandrel 295 results in plastic deformation of the folded portions 146 of the sheath 100 and provides a laid-over configuration of the folded/overlapping layers of the folded portions 146 as illustrated in FIG. 26 and FIGS. 27A-27C.

FIGS. 27A-27C illustrate various example folded structures resulting from the final compression step. It is contemplated that the number of folded portions 146 included on the sheath 100 is determined by the number of jaws 250 in the folding device 200 and the corresponding number of wings 284 provided on the mandrel 280. As provided in FIGS. 27A-27C, in some implementations the sheath 100 includes three, four or five folded portions 146. For example, in some implementations, as shown in FIG. 27A, the compressed distal end portion 140 of the sheath 100 includes at least three folded portions 146. In some implementations, as shown in FIG. 27B, the compressed distal end portion 140 of the sheath 100 includes four folded portions 146. In some implementations, as shown in FIG. 27C, the compressed distal end portion 140 of the sheath 100 includes five folded portions 146.

In some implementations, compressed air and a vacuum are optionally used to provide positive and negative pressure to the central lumen 112 of the sheath 100 during the folding/compression procedure. As described herein, applying sequentially positive and negative pressure to the central lumen 112 of the sheath 100 can help to ensure consistent folding of the folded portions of the sheath 100. When used, the opening at the distal end of the sheath 100 is sealed forming a sealed portion 160. In some examples, the sealed portion 160 is formed before the uncompressed sheath 100 is inserted into the folding device 200 as described herein in reference to FIG. 19. Further examples the sealed portion 160 is formed after the uncompressed sheath 100 is inserted into the folding device 200. In some examples, the sealed portion 160 is formed by a plug or other sealing member coupled at/to the opening at the distal end of the sheath 100. With the distal end of the sheath 100 sealed, a compressed air and vacuum supply device is coupled to the proximal end of the sheath 100. With the uncompressed sheath 100 received within the folding device 200, as illustrated for example in FIG. 19, a positive pressure is applied against to the central lumen 112/internal surface of the sheath 100. The jaws 250 are then partially rotated radially inward from the first position toward the second position, thereby partially creating the folded portions of the sheath 100. As described herein in reference to FIG. 20, and with a positive pressure applied against the central lumen 112/internal surface of the sheath 100, movement of the jaws 250 from the first position toward/to the second position, portions of the uncompressed sheath 100 are pressed against and captured between adjacent jaws thereby creating the folded portions of the sheath 100 and compressing the sheath 100. The jaws 250 are moved to/toward the second position until the desired fold is formed. It is contemplated that the compressed air supply device can be used in conjunction with the mandrel 280 and/or second mandrel 290 as described herein. That is, the positive pressure can be applied to the central lumen 112/inner surface of the sheath 100 with the mandrel 280 received within the central lumen 112 as described herein.

During the further compression steps, described herein in reference to FIG. 21 and FIGS. 24-26, a negative pressure is applied to the central lumen 112/inner surface of the sheath 100. For example, with the second mandrel 290 or third mandrel 295 positioned within the sheath 100, the positive pressure is realized, the compressed air/vacuum device switches to vacuum mode and creates negative pressure on the central lumen 112/inner surface of the sheath 100. The jaws 250 on the folding device 200 remain in the second position/closed position. Once a desired level of negative pressure is reached, the jaws 250 on the folding device 200 can be rotated to the first position, and the central opening 212/jaws 250 are opened. Thereafter, the folded sheath 100 can be detached from the folding device 200 and transferred, for example, to a conventional short crimper (for example, conventional sheath crimping mechanism 300) to complete the crimping and wrapping (pleating) process as previously described. The vacuum inside the sheath 100 will hold the initially formed folded portions 146 in the folded configuration while the sheath 100 is transferred into the working orifice of the crimper. In some examples, the negative pressure against the central lumen 112/internal surface of the sheath 100 helps the folded portions 146 to maintain their folded configuration. With the forming of the folded portions 146 of the sheath 100 complete, the sealed portion 160 can be removed.

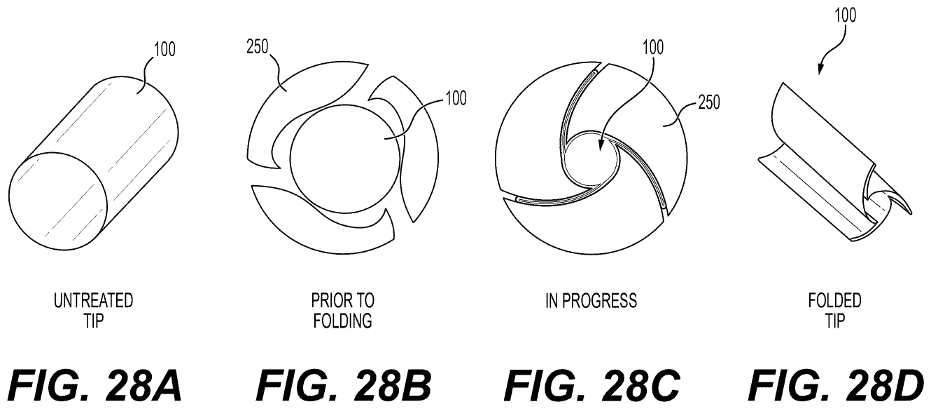

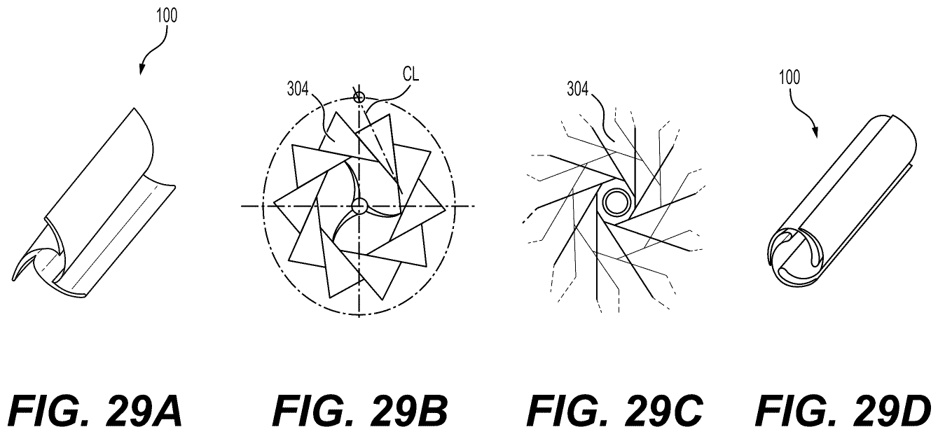

The folding process is represented schematically in FIGS. 28A-28D and FIGS. 29A-29D described herein. For example, as represented in FIGS. 28A and 28B, the uncompressed/unfolded portion of the sheath 100 is provided and inserted into the central opening 212 of the folding device 200. As represented in FIGS. 28B and 28C, jaws 250 are then rotated radially inward from the first position (retracted position) to/toward the second position (contracted position) such that at least a portion of the jaws 250 are displaced inward and portions of the uncompressed sheath 100 are captured between adjacent jaws 250. The portions of the sheath 100 captured between the jaw 250 are thereby formed into the folded portions 146 of the sheath 100 (see, for example, FIG. 28D).

The folded portions 146 of the sheath 100 are then compressed against the inner and outer surface of the sheath 100 in a pleating step. As represented in FIGS. 29A and 29B, the folded portion 146 of the sheath 100 are provided and advanced with the central opening 212 of the folding device 200 and/or within the compression channel 302 of a conventional sheath crimping mechanism 300. The folded portions 146 of the sheath 100 are then compressed against a supporting mandrel (see, for example, FIGS. 29B and 29C) as the plurality of jaws 304 of moved toward the second position. In some implementations, as illustrated in FIG. 29B, the center line (CL) of the plurality of jaws 304 is rotated toward the central axis of the compression channel 302. As such, the laid-over configuration of the folded layers/folded portions 146 is formed, as illustrated in FIG. 29D.

In some implementations, the movement of the jaws 250 of the folding device 200 between the first and second position is controlled and driven by an external source of inward and outward radial force.

In some implementations, movement of the jaws 250 of the folding device 200 is controlled and driven by a conventional sheath crimping mechanism 300. An example, conventional sheath crimping mechanism 300 is illustrated in FIG. 30. In some implementations, as illustrated in FIG. 21, the housing 210 of the folding device 200 is fitted within the compression channel 302 of a conventional sheath crimping mechanism 300 such that expansion and contraction of the diameter of the compression channel 302 drive the corresponding movement of the jaws 250 between the first and second position. As illustrated in FIG. 21, the crimping mechanism 300 includes a plurality of jaws 304 movable to contract and expand the diameter of a compression channel 302.

In some implementations, in the final folding/compression step, the inwardly directed radial force for folding the sheath 100 is optionally provided by a conventional sheath crimping mechanism 300, as illustrated in FIGS. 24 and 25. The folded/compressed distal end portion 140 of the sheath 100 is inserted within the compression channel 302 of the crimping mechanism 300. Expansion and contraction of the diameter of the compression channel are driven by the corresponding movements of a plurality of jaws 304.

In some implementations, a heat treatment is applied to the folded/compressed portion (for example, distal end portion 140) of the sheath 100 fixing the folded/compressed shape of the sheath 100. As illustrated in FIG. 31, the distal end portion 140 of the sheath 100 is compressed/folded as described herein. As shown in FIG. 31, the uncompressed/unfolded distal end portion 140 of the sheath 100 has a diameter corresponding to the diameter of the elongated body portion of the sheath 100. The distal end portion 140 of the sheath 100 of the sheath 100 is folded/compressed as described herein. In some implementations, a heat-shrink tubing 400 is optionally provided over the distal end portion 140 of the sheath 100. A heat process is applied to the sheath 100 bonding the various layers of the sheath 100, for example, inner layer 102 and outer layer 108, in the folded/compression configuration. The heat-shrink tubing can be removed resulting in the desired tip-shape shown in FIGS. 32-34. In some implementations, the sealed portion 160 is removed in conjunction with the heat-shrink tubing.

The heating temperature will be lower than the melting point of the material used. In some implementations, the heat shrink tube can have a melting point that is about the same as the melting point of the distal end portion 140 material. The sheath with the heat shrunk tube extending over the sheath 100 and the distal end portion 140 is heated again (for example, to about 125 degrees Celsius for sheaths including Dyneema® outer layers and distal end portions). In some examples, this causes the sheath to crimp to an even smaller diameter. At the distal end portion 140, a higher temperature can be applied (for example, from about 145 degrees Celsius to about 155 degrees Celsius for Dyneema® material) causing the layers of material to melt together in the folded configuration shown in FIGS. 33 and 34. The bonds at the distal end portion 140 induced by the high temperature melting step will still be weak enough to be broken by a passing delivery system. As a final step, the heat shrink tube is removed, and the shape of the sheath remains at the folded/crimped diameter.

Implementations of the sheaths described herein may comprise a variety of lubricious outer coatings, including hydrophilic or hydrophobic coatings, and/or surface blooming additives or coatings.

In some implementations, the distal end portion of the sheath (and/or of the vessel dilator) can decrease from the initial diameter of the sheath (for example, 8 mm) to 3.3 mm (10F), and may decrease to the diameter of a guide wire, allowing the sheath and/or the vessel dilator to run on a guide wire.

### General Considerations

For purposes of this description, certain aspects, advantages, and novel features of the implementations of this disclosure are described herein. The disclosed methods, apparatus, and systems should not be construed as being limiting in any way. Instead, the present disclosure is directed toward all novel and nonobvious features and aspects of the various disclosed implementations, alone and in various combinations and sub-combinations with one another. The methods, apparatus, and systems are not limited to any specific aspect or feature or combination thereof, nor do the disclosed implementations require that any one or more specific advantages be present or problems be solved.

Although the operations of some of the disclosed implementations are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the terms “coupled” and “associated” generally mean electrically, electromagnetically, and/or physically (for example, mechanically or chemically) coupled or linked and does not exclude the presence of intermediate elements between the coupled or associated items absent specific contrary language.

In the context of the present application, the terms “lower” and “upper” are used interchangeably with the terms “inflow” and “outflow”, respectively. Thus, for example, the lower end of a valve is its inflow end and the upper end of the valve is its outflow end.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device toward the user, while distal motion of the device is motion of the device away from the user. The terms “longitudinal” and “axial” refer to an axis extending in the proximal and distal directions, unless otherwise expressly defined.

Unless otherwise indicated, all numbers expressing dimensions, quantities of components, molecular weights, percentages, temperatures, forces, times, and so forth, as used in the specification or claims, are to be understood as being modified by the term “about.” Accordingly, unless otherwise indicated, implicitly or explicitly, the numerical parameters set forth are approximations that can depend on the desired properties sought and/or limits of detection under test conditions/methods familiar to those of ordinary skill in the art. When directly and explicitly distinguishing implementations from discussed prior art, the implementation numbers are not approximates unless the word “about” is recited. Furthermore, not all alternatives recited herein are equivalents.

### EXEMPLARY ASPECTS

In view of the many possible aspects to which the principles of the disclosed disclosure can be applied, it should be recognized that the illustrated aspects are only preferred examples of the disclosure and should not be taken as limiting the scope of the disclosure. Rather, the scope of the disclosure is defined by the following claims. We, therefore, claim as our disclosure all that comes within the scope and spirit of these claims.

Example 1: A folding device for an introducer sheath including: a housing including a central opening extending therethrough and a plurality of channels extending through a side wall of the housing, the plurality of channels positioned circumferentially around the housing; and a plurality of jaws rotatably coupled to the housing, each of the plurality of jaws is rotatable within a corresponding one of the plurality of channels, wherein each of the plurality of jaws are rotatable between a first position where the plurality of jaws do not extend into the central opening of the housing and a second position where at least a portion of each of the plurality of jaws extends into the central opening of the housing.

Example 2: The folding device according to any example herein, particularly example 1, wherein the central opening is sized and configured to receive an unfolded and/or uncompressed portion of an introducer sheath when the plurality of jaws are in the first position.

Example 3: The folding device according to any example herein, particularly examples 1-2, wherein the folding device is sized and configured to fold and/or collapse a circumference of a portion of a sheath positioned within the central opening of the housing when the plurality of jaws are moved from the first position to the second position such that symmetrical folds are formed around the circumference of the sheath.

Example 4: The folding device according to any example herein, particularly examples 1-3, wherein each of the plurality of jaws is rotatably coupled to the housing at a corresponding axel extending between a proximal end and distal end of the housing, each of the axels extending through a jaw axel bore provided in each of the plurality of jaws.

Example 5: The folding device according to any example herein, particularly example 4, wherein each of the axels extend through the housing at a corresponding housing axel bore.

Example 6: The folding device according to any example herein, particularly example 5, wherein the housing includes: a proximal end portion extending between a proximal end surface of the housing and a proximal end surface of each of the plurality of channels; and a distal end portion extending between a distal end surface of the housing and a distal end surface of each of the plurality of channels; wherein each of the housing axel bores extend through the proximal end portion and distal end portion of the housing.

Example 7: The folding device according to any example herein, particularly examples 1-6, wherein each of the plurality of jaws includes a first inner surface, a second inner surface, and an outer surface, where the second inner surface is located between the first inner surface and the outer surface.

Example 8: The folding device according to any example herein, particularly example 7, wherein the first inner surface defines a concave surface and the second inner surface defines a convex surface.

Example 9: The folding device according to any example herein, particularly examples 7-8, wherein a curvature of the outer surface corresponds with an outer curvature of the housing.

Example 10: The folding device according to any example herein, particularly examples 7-9, wherein, when the plurality of jaws are in the first position, the first inner surface is positioned proximate the central opening.

Example 11: The folding device according to any example herein, particularly examples 7-10, wherein, as the plurality of jaws move from the first position to the second position, the first inner surface and the second inner surface extend into the central opening of the housing.

Example 12: The folding device according to any example herein, particularly examples 1-11, wherein, as the plurality of jaws move from the first position to the second position, each of the plurality of jaws are displaced inward forming an opening between adjacent jaws.

Example 13: The folding device according to any example herein, particularly examples 1-12, wherein, as the plurality of jaws move from the first position to the second position, each of the plurality of jaws are displaced inward forming an opening between adjacent jaws.

Example 14: The folding device according to any example herein, particularly examples 1-13, wherein movement of the plurality of jaws to the second position results from displacing the plurality of jaws radially inward.

Example 15: The folding device according to any example herein, particularly examples 1-14, further including: a mandrel sized and configured to be received within the central opening when the plurality of jaws are in the second position.

Example 16: The folding device according to any example herein, particularly example wherein a portion of the mandrel has a star-shaped cross section.

Example 17: The folding device according to any example herein, particularly example 16, wherein the portion of the mandrel including the star-shaped cross section includes crescent-shaped wings extending at an angle radially outward from a body portion of the mandrel.

Example 18: The folding device according to any example herein, particularly example 15-17, further including a second mandrel sized and configured to be received with the central opening, the second mandrel having a diameter less than a diameter of the mandrel and having a circular-shaped cross section; and a third mandrel sized and configured to be received within the central opening, the third mandrel having a diameter less than a diameter of the second mandrel and having a circular-shaped cross section.

Example 19: The folding device according to any example herein, particularly example 1-18, further including a crimping mechanism for directing movement of the plurality of jaws between the first position and the section position, wherein the housing is received within the crimping mechanism.

Example 20: The folding device according to any example herein, particularly example 19, wherein the crimping mechanism includes a plurality of jaws movable to contract and expand a diameter of a compression channel provided within the crimping mechanism, wherein the housing is received within the compression channel such that expansion and contraction of the diameter of the compression channel drives a corresponding movement of the plurality of jaws between the first and second position.

Example 21: A method of folding a sheath comprising: inserting a portion of an uncompressed sheath into a folding device, the folding device including: a housing including a central opening extending therethrough and a plurality of channels extending through a side wall of the housing, the plurality of channels positioned circumferentially around the housing; and a plurality of jaws rotatably coupled to the housing, each of the plurality of jaws is rotatable within a corresponding one of the plurality of channels; wherein each of the plurality of jaws are rotatable between a first position where the plurality of jaws do not extend into the central opening of the housing and a second position where at least a portion of each of the plurality of jaws extends into the central opening of the housing; and moving the plurality of jaws from the first position toward the second position by rotating each of the plurality of jaws radially inward such that the portion of the uncompressed sheath is captured between adjacent jaws thereby creating folded portions of the sheath and forming a compressed portion of the sheath.

Example 22: The method of folding a sheath according to any example herein, particularly example 21, wherein the plurality of jaws are moved from the first position toward the second position in a coordinated manner.

Example 23: The method of folding a sheath according to any example herein, particularly examples 21-22, wherein the plurality of jaws are moved from the first position toward the second position in response to a radially inward force provided against an outer surface of the jaws.

Example 24: The method of folding a sheath according to any example herein, particularly examples 21-23, further including: inserting a mandrel within a central lumen of the uncompressed sheath, the mandrel having a star-shaped cross section including a plurality of wings extending radially outward from a body portion of the mandrel; and moving the plurality of jaws from the first position toward the second position by rotating each of the plurality of jaws radially inward such that the portion of the uncompressed sheath is captured between adjacent jaws and compressed against the plurality of wings provided on the mandrel, thereby creating the folded portions of the sheath disposed around its circumference and forming the compressed portion of the sheath.

Example 25: The method of folding a sheath according to any example herein, particularly example 24, wherein, in the second position, the plurality of wings define a shape complementary to a star-shaped opening between the plurality of jaws.

Example 26: The method of folding a sheath according to any example herein, particularly examples 24-25, further including: withdrawing the mandrel from the central lumen of the compressed portion of the sheath; and inserting a second mandrel within the central lumen of the compressed portion of the sheath, the second mandrel having a diameter less than a diameter of the mandrel.

Example 27: The method of folding a sheath according to any example herein, particularly example 26, moving the plurality of jaws toward the second position by rotating each of the plurality of jaws radially inward such that the folded portions of the sheath are further compressed against an outer surface of the second mandrel.

Example 28: The method of folding a sheath according to any example herein, particularly examples 26-27, further including: removing the sheath from the central lumen of the folding device and withdrawing the second mandrel from the central lumen of the portion of the sheath; and further compressing the compressed portion of the sheath.

Example 29: The method of folding a sheath according to any example herein, particularly example 28, further including: inserting the compressed portion of the sheath into the central lumen of the folding device; and further compressing the compressed portion of the sheath by moving the plurality of jaws toward the second position.

Example 30: The method of folding a sheath according to any example herein, particularly example 28, wherein the sheath is provided over a third mandrel, wherein further compressing the compressed portion of the sheath includes compressing the folded portions against an outer surface of the third mandrel.

Example 31: The method of folding a sheath according to any example herein, particularly example 30, wherein compression of the folded portions against the outer surface of the third mandrel results in plastic deformation of the folded portions of the sheath and provides a laid-over configuration of the folded portions.

Example 32: The method of folding a sheath according to any example herein, particularly examples 30-31, wherein the compressed portion of the sheath is further compressed using a crimping mechanism, where further compressing the compressed portion of the sheath includes inserting the compressed portion of the sheath within a compression channel of the crimping mechanism such that expansion and contraction of a diameter of the compression channel drives a corresponding movement of the plurality of jaws between the first and second position.

Example 33: The method of folding a sheath according to any example herein, particularly examples 21-32, where the housing is received within a compression channel of a crimping mechanism such that expansion and contraction of a diameter of the compression channel drives a corresponding movement of the plurality of jaws between the first and second position.

Example 34: The method of folding a sheath according to any example herein, particularly examples 21-33, further includes: applying a heat treatment to the compressed portion of the sheath.

Example 35: The method of folding a sheath according to any example herein, particularly examples 21-34, wherein the compressed portion of the sheath includes at least three folded portions.

Example 36: The method of folding a sheath according to any example herein, particularly examples 21-35, further including: sealing a distal end of the uncompressed sheath, forming a sealed portion; coupling a compressed air and vacuum device to the proximal end of the uncompressed sheath; applying a positive pressure from the compressed air and vacuum device against an internal surface of the sheath; moving the plurality of jaws toward the second position by rotating each of the plurality of jaws radially inward thereby creating partially folded portions of the sheath (for example, the first folding step, with star-shaped mandrel); and applying a negative pressure against the internal surface of the partially compressed sheath and further moving the plurality of jaws toward the second position by rotating each of the plurality of jaws radially inward thereby creating the folded portions of the sheath and forming the compressed portion of the sheath (for example, the second folding step, with second mandrel).

Example 37: The method of folding a sheath according to any example herein, particularly example 36, wherein the positive pressure is applied against the internal surface of the sheath before the plurality of jaws are moved from the first position toward the second position.

Example 38: The method of folding a sheath according to any example herein, particularly example 36, wherein the positive pressure is applied against the internal surface of the sheath after the plurality of jaws have partially moved from the first position toward the second position and the folded portions of the sheath have been partially formed.

Example 39: The method of folding a sheath according to any example herein, particularly examples 36-38, further including: further compressing the compressed portion of the sheath by moving the plurality of jaws toward the second position such that the folded portions plastically deform in a laid-over configuration; applying a heat treatment to the compressed portion of the sheath; and removing the sealed portion from the distal end of the sheath.

In view of the many possible implementations to which the principles of the disclosed technology may be applied, it should be recognized that the illustrated implementations are only preferred examples and should not be taken as limiting the scope of the disclosure. Rather, the scope of the disclosure is at least as broad as the following claims. We therefore claim all that comes within the scope and spirit of these claims

1. A folding device for an introducer sheath including:
a housing including a central opening extending therethrough and a plurality of channels extending through a side wall of the housing, the plurality of channels positioned circumferentially around the housing; and
a plurality of jaws rotatably coupled to the housing, each of the plurality of jaws is rotatable within a corresponding one of the plurality of channels,
wherein each of the plurality of jaws are rotatable between a first position where the plurality of jaws do not extend into the central opening of the housing and a second position where at least a portion of each of the plurality of jaws extends into the central opening of the housing.

2. The folding device of claim 1, wherein the central opening is sized and configured to receive an unfolded and/or uncompressed portion of an introducer sheath when the plurality of jaws are in the first position, and
wherein the folding device is sized and configured to fold and/or collapse a circumference of a portion of a sheath positioned within the central opening of the housing when the plurality of jaws are moved from the first position to the second position such that symmetrical folds are formed around the circumference of the sheath.

3. The folding device of claim 1, wherein each of the plurality of jaws is rotatably coupled to the housing at a corresponding axel extending between a proximal end and distal end of the housing, each of the axels extending through a jaw axel bore provided in each of the plurality of jaws.

4. The folding device of claim 3, wherein each of the axels extend through the housing at a corresponding housing axel bore,
wherein the housing further comprises:
a proximal end portion extending between a proximal end surface of the housing and a proximal end surface of each of the plurality of channels; and
a distal end portion extending between a distal end surface of the housing and a distal end surface of each of the plurality of channels;
wherein each of the housing axel bores extend through the proximal end portion and distal end portion of the housing.

5. The folding device of claim 1, wherein each of the plurality of jaws includes a first inner surface, a second inner surface, and an outer surface, where the second inner surface is located between the first inner surface and the outer surface,
wherein the first inner surface defines a concave surface and the second inner surface defines a convex surface, and
wherein a curvature of the outer surface corresponds with an outer curvature of the housing.

6. The folding device of claim 5, wherein, when the plurality of jaws are in the first position, the first inner surface is positioned proximate the central opening, and
wherein, as the plurality of jaws move from the first position to the second position, the first inner surface and the second inner surface extend into the central opening of the housing.

7. The folding device of claim 1, wherein, as the plurality of jaws move from the first position to the second position, each of the plurality of jaws are displaced inward forming an opening between adjacent jaws, and
wherein movement of the plurality of jaws to the second position results from displacing the plurality of jaws radially inward.

8. The folding device of claim 1, further including:
a mandrel sized and configured to be received within the central opening when the plurality of jaws are in the second position, a portion of the mandrel having a star-shaped cross section including crescent-shaped wings extending at an angle radially outward from a body portion of the mandrel.

9. The folding device of claim 8, further including:
a second mandrel sized and configured to be received with the central opening, the second mandrel having a diameter less than a diameter of the mandrel and having a circular-shaped cross section; and
a third mandrel sized and configured to be received within the central opening, the third mandrel having a diameter less than a diameter of the second mandrel and having a circular-shaped cross section.

10. The folding device of claim 1, further including:
a crimping mechanism for directing movement of the plurality of jaws between the first position and the second position,
wherein the housing is received within the crimping mechanism,
wherein the crimping mechanism includes a plurality of jaws movable to contract and expand a diameter of a compression channel provided within the crimping mechanism, wherein the housing is received within the compression channel such that expansion and contraction of the diameter of the compression channel drives a corresponding movement of the plurality of jaws between the first and second position.

11. A method of folding a sheath comprising:
inserting a portion of an uncompressed sheath into a folding device, the folding device including:
a housing including a central opening extending therethrough and a plurality of channels extending through a side wall of the housing, the plurality of channels positioned circumferentially around the housing; and
a plurality of jaws rotatably coupled to the housing, each of the plurality of jaws is rotatable within a corresponding one of the plurality of channels;
wherein each of the plurality of jaws are rotatable between a first position where the plurality of jaws do not extend into the central opening of the housing and a second position where at least a portion of each of the plurality of jaws extends into the central opening of the housing; and
moving the plurality of jaws from the first position toward the second position by rotating each of the plurality of jaws radially inward such that the portion of the uncompressed sheath is captured between adjacent jaws thereby creating folded portions of the sheath and forming a compressed portion of the sheath.

12. The method of claim 11, wherein the plurality of jaws are moved from the first position toward the second position in response to a radially inward force provided against an outer surface of the jaws.

13. The method of claim 11, further including:
inserting a mandrel within a central lumen of the uncompressed sheath, the mandrel having a star-shaped cross section including a plurality of wings extending radially outward from a body portion of the mandrel; and
moving the plurality of jaws from the first position toward the second position by rotating each of the plurality of jaws radially inward such that the portion of the uncompressed sheath is captured between adjacent jaws and compressed against the plurality of wings provided on the mandrel, thereby creating the folded portions of the sheath disposed around its circumference and forming the compressed portion of the sheath,
wherein, in the second position, the plurality of wings define a shape complementary to a star-shaped opening between the plurality of jaws.

14. The method of claim 13, further including:
withdrawing the mandrel from the central lumen of the compressed portion of the sheath;
inserting a second mandrel within the central lumen of the compressed portion of the sheath, the second mandrel having a diameter less than a diameter of the mandrel;
moving the plurality of jaws toward the second position by rotating each of the plurality of jaws radially inward such that the folded portions of the sheath are further compressed against an outer surface of the second mandrel;
removing the sheath from the central lumen of the folding device and withdrawing the second mandrel from the central lumen of the portion of the sheath; and
further compressing the compressed portion of the sheath.

15. The method of claim 14, further including:
inserting the compressed portion of the sheath into the central lumen of the folding device; and
further compressing the compressed portion of the sheath by moving the plurality of jaws toward the second position.

16. The method of claim 14, wherein the sheath is provided over a third mandrel,
wherein further compressing the compressed portion of the sheath includes compressing the folded portions against an outer surface of the third mandrel such that compression of the folded portions against the outer surface of the third mandrel results in plastic deformation of the folded portions of the sheath and provides a laid-over configuration of the folded portions.

17. The method of claim 16, wherein the compressed portion of the sheath is further compressed using a crimping mechanism, where further compressing the compressed portion of the sheath includes inserting the compressed portion of the sheath within a compression channel of the crimping mechanism such that expansion and contraction of a diameter of the compression channel drives a corresponding movement of the plurality of jaws between the first and second position.

18. The method of claim 11, wherein the housing is received within a compression channel of a crimping mechanism such that expansion and contraction of a diameter of the compression channel drives a corresponding movement of the plurality of jaws between the first and second position.

19. The method of claim 11, further including:
sealing a distal end of the uncompressed sheath, forming a sealed portion;
coupling a compressed air and vacuum device to a proximal end of the uncompressed sheath;
applying a positive pressure from the compressed air and vacuum device against an internal surface of the sheath;
moving the plurality of jaws toward the second position by rotating each of the plurality of jaws radially inward thereby creating partially folded portions of the sheath; and
applying a negative pressure against the internal surface of the partially compressed sheath and further moving the plurality of jaws toward the second position by rotating each of the plurality of jaws radially inward thereby creating the folded portions of the sheath and forming the compressed portion of the sheath.

20. The method of claim 19, further including:
further compressing the compressed portion of the sheath by moving the plurality of jaws toward the second position such that the folded portions plastically deform in a laid-over configuration;
applying a heat treatment to the compressed portion of the sheath; and
removing the sealed portion from the distal end of the sheath.
